(19) United States
(12) Patent Application Publication
(10) Pub. No.: US 2021/0382071 A1
(43) Pub. Date: Dec. 9, 2021

(54) BIOMARKERS FOR MITOCHONDRIAL DISEASES AND RELATED METHODS

(71) Applicant: HELSINGIN YLIOPISTO, Helsingin Yliopisto (FI)

(72) Inventors: Anu WARTIOVAARA, Helsinki (FI); Jana BUZKOVA, Helsinki (FI)

(21) Appl. No.: 17/284,129

(22) PCT Filed: Oct. 9, 2019
(86) PCT No.: PCT/FI2019/050718

§ 371 Date: Apr. 9, 2021

(30) Foreign Application Priority Data
Oct. 10, 2018 (FI) 20185846

(51) Int. Cl. G01N 33/68; G01N 33/66

(52) U.S. Cl. CPC G01N 33/6896; G01N 33/66; G01N 33/6812; G01N 33/6815; G01N 2800/10; G01N 33/6893; G01N 2800/52; G01N 2800/2835; G01N 33/6848

(57) ABSTRACT

The present invention relates to the fields of life sciences and medicine. Specifically, the invention relates to a method for determining a mitochondrial disorder of a subject or predicting a prognosis of a subject having a mitochondrial disorder, wherein the method comprises determining specific biomarkers from a sample of a subject. Also, the present invention relates to a method of selecting a treatment for a subject having a mitochondrial disorder or following up a treatment of a subject having a mitochondrial disorder, wherein the method comprises determining specific biomarkers from a sample of a subject. Still, the present invention relates to a kit comprising tools for determining said specific biomarkers from a sample of a subject and to use of the kit or specific biomarkers of the present invention for determining a mitochondrial disorder of a subject, predicting a prognosis of a subject having a mitochondrial disorder, selecting a treatment for a subject having a mitochondrial disorder or following up a treatment of a subject having a mitochondrial disorder.

### FIELD OF THE INVENTION

The present invention relates to the fields of life sciences and medicine. Specifically, the invention relates to a method for determining a mitochondrial disorder of a subject or predicting a prognosis of a subject having a mitochondrial disorder, wherein the method comprises determining specific biomarkers from a sample of a subject. Also, the present invention relates to a method of selecting a treatment for a subject having a mitochondrial disorder or following up a treatment of a subject having a mitochondrial disorder, wherein the method comprises determining specific biomarkers from a sample of a subject. Still, the present invention relates to a kit comprising tools for determining said specific biomarkers from a sample of a subject and to use of the kit or specific biomarkers of the present invention for determining a mitochondrial disorder of a subject, predicting a prognosis of a subject having a mitochondrial disorder, selecting a treatment for a subject having a mitochondrial disorder or following up a treatment of a subject having a mitochondrial disorder.

### BACKGROUND OF THE INVENTION

Mitochondrial disorders are inherited multi-organ diseases with variable phenotypes. Mitochondrial disorders are the most common group of inherited metabolic diseases, with exceptional clinical variability. Globally, their minimum birth prevalence is 1 in 2000-5000 individuals (Gorman et al. 2016; Thorburn 2004). The adult forms present most commonly with neurological or muscular symptoms (Suomalainen 2011), but their diagnosis is challenging, and treatment options are scarce. Furthermore, the molecular mechanisms of tissue-specificity and clinical variability in mitochondrial disorders are unknown.

Mitochondrial dysfunction is also a characteristic sign of inclusion body myositis (IBM), which is a sporadic inflammatory muscle disease, the most common acquired myopathy in the elderly with a prevalence of 2-4: 100,000 in Nordic countries (Lindgren, Lindberg, and Oldfors 2017).

In some patient cases the diagnosis of mitochondrial disorders can be confirmed by identification of a pathogenic gene variant by genetic testing of DNA extracted from a blood sample. However, in many individuals further approaches such as family history, blood and/or CSF lactate concentration, neuroimaging, tissue sampling by biopsy to study histology and mitochondrial functions, cardiac evaluation, and molecular genetic testing for a nuclear gene pathogenetic variant are needed. If genetic testing does not reveal a disease, further clinical tests may be carried out.

Now, e.g. serum markers lactate and pyruvate used for determining mitochondrial diseases are not very specific and sensitive. Furthermore, there are no available serum markers or genetic testing for IBM. Thus, there remains a significant unmet need for effective, specific and sensitive methods for diagnosing mitochondrial disorders or for determining a patient having an increased risk for a mitochondrial disorder.

### BRIEF DESCRIPTION OF THE INVENTION

The objects of the invention are achieved by utilizing a specific combination of biomarkers for determining a subject with a mitochondrial disorder. Indeed, the present invention provides a simple method, which can be utilized either alone or together e.g. with clinical diagnostic methods for detecting mitochondrial disorders. The disease-specific metabolic biomarkers presented in this disclosure are valuable for diagnosing various disorders caused by dysfunctional mitochondria.

The present invention also concerns disease-specific metabolomic fingerprints present in samples (e.g. the blood, urine and muscle) of patients with different primary or secondary mitochondrial disorders. Thus, the present invention also reveals pathogenic pathways and/or potential treatment targets. A specific treatment may also be selected based on the biomarker test of the present invention for a subject having a mitochondrial disease. The disease-specific metabolic fingerprints are excellent tools for follow-up of disease progression and treatment effect. The specific combination of biomarkers of the present invention may be utilized e.g. for selecting a patient to specific treatment of a mitochondrial disorder or following up treatment of a subject having a mitochondrial disorder.

The present invention makes it possible e.g. to provide or find effective treatments for a specific subgroup of patients with disease-specific metabolomic fingerprints, and to reduce the time, work load and cost used for diagnosis. The sooner the patients with a mitochondrial disease are found the faster the treatment can be started. Indeed, the present invention solves the problems of conventional slow and unspecific methods for determining mitochondrial disorders.

An object of the present invention is thus to provide a tool and a method for effective as well as specific and sensitive detection and/or treatment of mitochondrial disorders.

The present invention relates to a method for determining a mitochondrial disorder of a subject or predicting a prognosis of a subject having a mitochondrial disorder, wherein the method comprises determining at least four biomarkers sorbitol, alanine, myoinositol and cystathionine from a sample of a subject.

Also, the present invention relates to a method of selecting a treatment for a subject having a mitochondrial disorder or following up a treatment of a subject having a mitochondrial disorder, wherein the method comprises determining at least four biomarkers sorbitol, alanine, myoinositol and cystathionine from a sample of a subject.

Still, the present invention relates to a kit (e.g. for determining a mitochondrial disorder, predicting a prognosis of a subject having a mitochondrial disorder, selecting a treatment for a subject having a mitochondrial disorder or following up a treatment of a subject having a mitochondrial disorder) comprising tools for determining at least four biomarkers sorbitol, alanine, myoinositol and cystathionine from a sample of a subject, and optionally reagents for performing a test (or an assay).

Still furthermore, the present invention relates to use of the kit of the present invention for determining a mitochondrial disorder of a subject, predicting a prognosis of a subject having a mitochondrial disorder, selecting a treatment for a subject having a mitochondrial disorder or following up a treatment of a subject having a mitochondrial disorder.

And still furthermore, the present invention relates to use of at least four biomarkers sorbitol, alanine, myoinositol and cystathionine for determining a mitochondrial disorder of a subject, predicting a prognosis of a subject having a mitochondrial disorder, selecting a treatment for a subject having a mitochondrial disorder or following up a treatment of a subject having a mitochondrial disorder.

Other objects, details and advantages of the present invention will become apparent from the following drawings, detailed description and examples.

### BRIEF DESCRIPTION OF THE DRAWINGS

FIGS. 1A-D show metabolomic fingerprints of primary mitochondrial diseases. A-D: Clustering of metabolome data in patients and controls; partial least squares discriminant analysis (PLS-DA) plots; variable importance in projection (VIP) score plots of top 15 metabolites; volcano plots of all metabolites in blood of infantile-onset spinocerebellar ataxia (IOSCA, A), mitochondrial recessive ataxia syndrome (MIRAS, B), progressive external ophthalmoplegia (PEO, C), mitochondrial myopathy, encephalopathy, lactic acidosis and stroke-like episodes (MELAS)/maternally inherited diabetes and deafness (MIDD, D). ^{a}Significantly changed metabolites outside the false-discovery-rate (FDR) cut-off. ^{b}Metabolites not significantly changed between patients and controls. Colours in VIP score and volcano plots indicate the same most relevant and/or significantly changed metabolites among all patient groups. C3, component 3; CDCA, chenodeoxycholic acid; GABA, γ-aminobutyric acid; HIAA, 5-Hydroxyindole-3-acetic acid; OH-Kyn, hydroxyl-DL-kynurenine; SDMA, symmetric dimethylarginine; TCA, taurocholic acid.

FIGS. 2A-C show metabolomic fingerprints of inclusion body myositis, muscle disorders of non-mitochondrial origin and MIRAS carriers. A-C Clustering of metabolome data in patients and controls; PLS-DA plots; VIP score plots of top 15 metabolites; volcano plots of all metabolites in blood of inclusion body myositis (IBM; A), non-mitochondrial neuromuscular disease patients (NMDs; B) and MIRAS carriers (C). ^{a}Significantly changed metabolites outside the false-discovery-rate (FDR) cut-off. ^{b}Metabolites not significantly changed between patients and controls. Colours in VIP score and volcano plots indicate the same most relevant and/or significantly changed metabolites among all patient groups. cAMP, cyclic adenosine monophosphate; C3, component 3; CDCA, chenodeoxycholic acid; GABA, γ-aminobutyric acid; HIAA, 5-Hydroxyindole-3-acetic acid; IMP, inosine monophosphate; OH-Kyn, hydroxyl-DL-kynurenine; OH-Trp, hydroxytryptophan; SDMA, symmetric dimethylarginine.

FIG. 3 shows results of quantification of disease-specific single metabolites in blood. (A) Relative values of single metabolites and creatine/creatinine ratios in blood of primary mitochondrial disease, IBM and NMD patients, and MIRAS carriers compared to controls. (B) Relative values of single metabolites in blood of adult IOSCA (marked “IOSCA”) patients and one IOSCA child patient compared to controls. Data information: All data represent mean±SD. For individual metabolites: *P<0.05, **P<0.01, ***P<0.001 (two sample T-test). For creatine/creatinine (cr/crn) ratio: *P=0.022, **P=0.005, ***P=0.0001 (Mann-Whitney test).

FIG. 4 shows muscle metabolomes of MIRAS, PEO and MELAS patients. A-B: Metabolomes of muscle of MIRAS (A) and PEO (B) patients; PLS-DA plots; VIP score plots of top 15 metabolites; volcano plots of all metabolites. ^{a}Significantly changed metabolites outside the FDR cut-off. ^{b}Metabolites not significantly changed between patients and controls. C: Methyl cycle, transsulfuration and glutathione biosynthesis pathways changed in IOSCA, MIRAS, PEO and MELAS patients. Circled text: metabolites changed in blood; red, increased; blue, decreased. Coloured text: metabolites changed in muscle; red, increased; blue, decreased. Selected for MELAS muscle (n=2) were metabolites with the highest fold-change. D: Relative values of metabolites in MIRAS, PEO and MELAS patients. AMP, adenosine monophosphate; car., carnitine; Hcy, homocysteine; NAD, nicotinamide adenine dinucleotide; TCA, taurocholic acid; UDP, uridine diphosphate. All data represent mean mean±SD. All data represent mean±SD. *P=0.031, **P=0.008 (two sample T-test).

FIG. 5 shows results of pathway analysis of blood and muscle metabolites. A-F: Changed metabolic pathways in blood of IOSCA (A), MIRAS (B), PEO (C), MELAS/MIDD (D), IBM (E) and NMD (F) patients. G,H: Changed metabolic pathways in muscle of PEO (G) and MIRAS (H) patients. Data information: Top 10 pathways with ≥10% of detected metabolites per pathway are shown. *5% metabolite coverage in the pathway. bio., biosynthesis; deg., degradation; metab., metabolism.

FIG. 6 shows blood metabolites as biomarkers for mitochondrial diseases. A: Receiver operating characteristic (ROC) curves for individual metabolites sorbitol, alanine, myoinositol and cystathionine (left) and conventional blood biomarkers lactate and pyruvate, and cytokine FGF21 (right) in blood of MIRAS, PEO and MELAS/MIDD patients (n=20) compared to controls (n=30). ROC analysis: AUC of sorbitol 0.81 (95% CI 0.68-0.94, P=0.0003), alanine 0.81 (95% CI 0.66-0.94, P=0.0003), myoinositol 0.79 (95% CI 0.66-0.91, P=0.0007) and cystathionine 0.78 (95% CI 0.65-0.91, P=0.001). AUC of conventional biomarkers: lactate 0.86 (95% CI 0.76-0.97, P=0.0001) and pyruvate 0.78 (95% CI 0.64-0.93, P=0.0017), and FGF21 0.87 (95% CI 0.74-0.99, P=0.0001). B: ROC curve for the combined “multi-biomarker” of sorbitol/alanine/myoinositol/cystathionine for primary mitochondrial disorders compared to controls (left); mean centroids for mitochondrial disorders, IBM and NMD patients, and MIRAS carriers compared to controls (right). AUC of “multi-biomarker” 0.94 (95% CI 0.89-0.995, P=0.0001). All data represent mean±SD. **P<0.01, ***P<0.001.

### DETAILED DESCRIPTION OF THE INVENTION

Mitochondria are responsible for creating more than 90% of the energy needed by the body to sustain life and support organ function. When mitochondria fail, less energy is generated within the cell causing cell injury and cell death. Mitochondrial diseases result from failures of the mitochondria. As used herein “mitochondrial disorders” are a clinically heterogeneous group of disorders that arise as a result of either inherited or spontaneous mutations in mitochondrial DNA (mtDNA) or nuclear DNA (nDNA) which lead to altered functions of the proteins or RNA molecules that normally reside in mitochondria or are associated with mitochondrial function. Gene defects may be inherited maternally, or in an autosomal recessive, dominant or Xlinked manner. Mitochondrial disorders may present at any age and may affect a single organ or multiple organs. Some individuals with a mutation in mtDNA or nDNA display clinical features falling within a clinical syndrome. However, disease phenotypes may greatly vary and thus many individuals do not fit into a specific clinical form. Because mitochondria perform many different functions in different tissues, they cause several different mitochondrial diseases. Symptoms of mitochondrial disorders may include but are not limited to one or more of the following: ptosis, external ophthalmoplegia, proximal myopathy and exercise intolerance, cardiomyopathy, sensorineural deafness, optic atrophy, pigmentary retinopathy, diabetes mellitus, fluctuating encephalopathy, seizures, dementia, migraine, stroke-like episodes, strokes, severe developmental delays, inability to walk, talk, see, or digest food, ataxia, spasticity, mid- and late pregnancy loss.

In one embodiment of the invention the mitochondrial disorder is a primary or secondary mitochondrial disorder. As used herein “a primary disorder” refers to a disorder that is caused by a genetic defect affecting mitochondrial function, and is opposed to “a secondary disorder”, where mitochondrial dysfunction is prominent but not the primary cause of the disease. In one embodiment of the invention mitochondrial disorders include but are not limited to one or more of the following: mitochondrial myopathy, mitochondrial cardiomyopathy, mitochondrial DNA translation disease, mitochondrial DNA expression disease, Mitochondrial DNA deletion disease, mitochondrial DNA depletion syndrome, infantile-onset spinocerebellar ataxia (IOSCA), Leber's hereditary optic neuropathy (LHON), Pyruvate dehydrogenase complex deficiency (PDCD), Autosomal Dominant Optic Atrophy (ADOA), Kearns-Sayre syndrome (KSS), progressive external ophthalmoplegia (PEO), chronic progressive external ophthalmoplegia (CPEO), Mitochondrial myopathy, Carnitine palmitoyltransferase I (CPT I) Deficiency, CPT II Deficiency, mitochondrial encephalomyopathy with lactic acidosis and stroke-like episodes (MELAS), myoclonic epilepsy with ragged-red fibers (MERRF), neurogenic weakness with ataxia and retinitis pigmentosa (NARP), Leigh syndrome (LS), Luft Disease, mitochondrial recessive ataxia syndrome (MIRAS), Alpers-Huttenlocher syndrome (AHS), Barth Syndrome or LIC (Lethal Infantile Cardiomyopathy), beta-oxidation defects, carnitine-acyl-carnitine deficiency, carnitine deficiency, creatine deficiency syndromes, co-enzyme Q10 deficiency, complex I deficiency, complex II deficiency, complex III deficiency, complex IV deficiency or cytochrome C-oxidase (COX) deficiency, complex V deficiency, lactic acidosis, leukoencephalopathy with brain stem and spinal cord involvement and lactate elevation (LBSL)—leukodystrophy, long-chain acyl-CoA dehydrogenase deficiency (LOAD), long-chain 3-hydroxyacyl-CoA dehydrogenase deficiency (LCHAD), multiple acyl-CoA dehydrogenase deficiency (MAD) or glutaric aciduria type II, medium-chain acyl-CoA dehydrogenase deficiency (MCAD), mitochondrial cytopathy, mitochondrial DNA depletion, mitochondrial encephalopathy, a defect of mitochondrial translation, maternally inherited diabetes and deafness (MIDD), mitochondrial neurogastrointestinal disorder and encephalopathy (MNGIE), Pearson syndrome, pyruvate carboxylase deficiency, pyruvate dehydrogenase deficiency, POLG mutations, short-chain acyl-CoA dehydrogenase deficiency (SCAD), encephalopathy and possibly liver disease or cardiomyopathy (SCHAD), very long-chain acyl-CoA dehydrogenase deficiency (VLCAD), Friedreich's ataxia, Parkinson's disease, inclusion body myositis (IBM).

In one embodiment of the invention the primary mitochondrial disorder is a dysfunction affecting the skeletal muscle, heart, central and peripheral nervous system, liver, kidney, and/or the sensory organ systems (such as eye and ear).

In another embodiment of the invention the primary mitochondrial disorder is selected from the group consisting of mtDNA expression disorders: mitochondrial myopathy, mitochondrial cardiomyopathy, mitochondrial encephalopathy, mitochondrial hepatopathy, mitochondrial renal disease, mitochondrial intestinal disease, mitochondrial blood disease, mitochondrial DNA translation disease, mitochondrial DNA deletion disease, mitochondrial DNA depletion syndrome (including its different tissue-specific forms, for example but not limited by muscle-specific, brain-liver or heart specific mtDNA depletion syndrome), infantile-onset spinocerebellar ataxia (IOSCA), mitochondrial recessive ataxia syndrome (MIRAS), progressive external ophthalmoplegia (PEO), chronic progressive external ophthalmoplegia (CPEO), myoclonic epilepsy and ragged-red fibers (MERRF), Kearns-Sayre syndrome (KSS), and a defect of mitochondrial translation such as mitochondrial encephalomyopathy, lactic acidosis and stroke-like episodes (MELAS) or maternally inherited diabetes and deafness (MIDD), including non-symptomatic carriers of disease alleles.

In one embodiment of the invention the secondary mitochondrial disorder is an inclusion body myositis (IBM) or Parkinson's disease. IBM is a sporadic inflammatory muscle disease, which also shows mitochondrial dysfunction and multiple mtDNA deletions in the skeletal muscle, in addition to inflammatory changes. The pathogenic mechanism of sporadic IBM, the inflammatory and treatment resistant muscle disease, is still unknown, although it is one of the most frequently encountered muscle diseases in neurology clinics. Typical findings include inflammation, increased number of autophagosomes, and characteristics of mitochondrial myopathy: respiratory chain deficient muscle fibers and accumulation of multiple mtDNA deletions (Oldfors et al. 1995). These mitochondrial changes are considered to be a secondary consequence of IBM pathogenesis, probably due to insufficient turnover of mitochondria as a result of insufficient macroautophagy/mitophagy (Askanas, Engel, and Nogalska 2015), but whether they have functional consequences has been unknown. Parkinson's disease is a neurodegenerative disorder of adult age, inherited or sporadic. These patients show respiratory chain deficient neurons and neuron loss most typically in substantia nigra region of the brain, and have increased amounts of mtDNA deletions in the brain (Kraytsberg et al. and Bender et al.). The pathogenic changes are considered secondary to the pathogenesis of Parkinson's disease, but to contribute to disease progression. No blood biomarkers exist for the disease.

In the study of the present disclosure it was investigated whether primary and secondary mitochondrial disorders modify metabolism to reveal pathogenic pathways and biomarkers. Metabolomes of 25 mitochondrial myopathy or ataxias patients, 16 unaffected carriers, six IBM and 15 non-mitochondrial neuromuscular diseases (NMD) patients and 30 matched controls were investigated.

In the present study the applicants report disease-specific metabolomic fingerprints of primary mitochondrial disorders (including but not limited to mitochondrial muscle and brain disorders), secondary mitochondrial disorders (e.g. inclusion body myositis) and a mixed group of severe primary muscle dystrophies/atrophies. The present invention shows the following: 1) All the disease groups show blood metabolic fingerprints that cluster separately from healthy controls, indicating the potential of these fingerprints as multi-biomarkers for diagnosis, disease progression and treatment effect; 2) Secondary mitochondrial disorders (e.g. IBM) cause similar global metabolomic changes as primary mitochondrial myopathies (e.g. reflected in blood), revealing that metabolic strategies for intervention may be shared in these disease groups; 3) Heterozygous carriership for the recessive MIRAS allele, common in Western populations (Hakonen et al. 2005; Winterthun et al. 2005; population frequency 1:84 in Finns and 1:100 in Norwegians; www.sisuproject.fi) is not metabolically neutral; 4) The present omics approach identified known therapy targets in clinical use (e.g. arginine in MELAS/MIDD blood and muscle (Koga et al. 2005; Koenig et al. 2016); creatine in NMDs (Kley, Tarnopolsky, and Vorgerd 2013)) and identified new targets for treatment of mitochondrial disorders (e.g. IOSCA (creatine, glutathione [N-acetyl-cysteine] and NAD⁺[nicotinamide riboside] supplementation) or IBM (creatine supplementation)), proposing that targeted metabolomics analysis of metabolome may not only be valuable for mechanistic studies, but also suggest metabolic targets for treatment trials.

The present finding of the similarity of blood metabolomes of the primary and secondary (e.g. IBM) mitochondrial disorders suggests that the mitochondrial dysfunction drives the metabolic changes in secondary mitochondrial disorders (e.g. IBM) reflected in the blood.

A prominent metabolic pattern in different mitochondrial disorders in blood and muscle pointed to aberrant folate driven 1-carbon (1C)-cycle, which is the major cellular anabolic biosynthesis pathway, providing 1C-units for growth and repair. The pathways that feed from this cycle depend on cell-type needs and include de novo purine synthesis, methyl cycle, genome and metabolite methylation (creatine and phospholipid synthesis) and transsulfuration (cysteine metabolism, glutathione and taurine synthesis). The most prominent hits in IOSCA, MIRAS, PEO, MELAS and IBM pointed to aberrant transsulfuration pathway, with the most significant depletion of taurine and reduced form of glutathione found in IOSCA. Related findings were observed also in muscle of MIRAS patients, with more emphasis in the proximal folate-pool and methyl cycle: low methyl-donor S-Adenosyl-methionine and high S-Adenosylhomocysteine point to lowered methylation capacity. The activation of polyol pathway (sorbitol, myoinositol), which is a sign of high glucose uptake in the muscle, is also known to challenge regeneration of reduced glutathione (Brownlee 2001). These changes in mtDNA maintenance diseases, most prominently in IOSCA, point to a challenged glutathione supply, and suggest that N-acetyl-cysteine supplementation, providing cysteine for glutathione and taurine synthesis, could be tried as a metabolic bypass therapy.

Unbiased screen of the present study identified creatine depletion in NMD patients. Similarly low global creatine pool, represented by the blood creatine/creatinine ratio, was found to be present in IBM and also in IOSCA.

Omics approach of the present study highlighted a deficiency of arginine to be specific for MELAS/MIDD both in blood and muscle, as the only significantly decreased amino acid.

In the present study the full set of ˜100 metabolites was found very informative. Also, the present invention revealed a minimal set of four individual metabolites that were enough to distinguish mitochondrial disorders from other muscle-manifesting disorders as a “multi-biomarker”: cystathionine, sorbitol, myoinositol and alanine. Sorbitol and myoinositol have not been reported previously to be changed in mitochondrial disorders. Elevated cystathionine was found in single patients with mtDNA depletion syndrome (Mudd et al. 2012; Tadiboyina et al. 2005), but not in blood samples of patients with Leigh syndrome (Thompson Legault et al. 2015), caused by a structural defect of the respiratory chain. Alanine is a standard blood biomarker in mitochondrial disorders (Haas et al. 2008), but is also found increased in other conditions, including sepsis, tetraspasticity, hyperinsulinism, chronic thiamine deficiency, or as a side effect of valproic acid treatment (Morava et al. 2006; Noguera et al. 2004; Thabet et al. 2000; Thauvin-Robinet et al. 2004). Despite lacking sensitivity as single metabolites, their power increases as a combined multi-biomarker.

Also, said multi-biomarker can be utilized in follow-up of disease progression and therapy effect, e.g. when testing of a large targeted metabolome is not feasible.

Increased carbohydrate metabolites, but not cystathionine and/or alanine, were detected in blood of asymptomatic MIRAS carriers.

Arginine and proline metabolism pathways as well as pathways involving glutamate were found as the top changed pathways in the blood metabolomes of our NMD patients. These findings show that a semi-quantitative metabolomics assay—or arginine/proline content of serum—is useful as a multi-biomarker for treatment follow-up in muscle dystrophies.

In summary, in the study of the present disclosure mitochondrial disorder and IBM metabolomes clustered separately from controls and NMDs. Mitochondrial disorders and IBM showed transsulfuration pathway changes, creatine and niacinamide depletion marked NMDs, IBM and IOSCA. Low blood and muscle arginine was specific for MELAS/MIDD. A four-metabolite blood multi-biomarker (sorbitol, alanine, myoinositol, cystathionine) distinguished primary mitochondrial disorders from others (76% sensitivity, 95% specificity). The present omics approach identified pathways currently used to treat NMDs and mitochondrial stroke-like episodes and proposes nicotinamide riboside in mitochondrial disorders and IBM, and creatine in IOSCA and IBM as novel treatment targets. Importantly, the present omics screen identified targets for metabolite treatment, both verifying previously known targets and suggesting novel ones for IOSCA and IBM, disorders with few treatment options.

The results of the present study are highlighting the potential of targeted metabolomics of patient samples for mechanistic studies and/or as biomarkers for follow-up of disease progression and treatment effects.

Metabolome refers to the complete set of small-molecule metabolites (such as metabolic intermediates, hormones and other signaling molecules, and secondary metabolites) to be found within a biological sample, such as a single organism. Metabolites are the intermediates and products of metabolism and are defined herein as molecules less than 1 kDa in size. Examples of small-molecule metabolites include but are not limited to lipids, alcohols, nucleotides, organic acids, antioxidant molecules, sugar derivatives, vitamins and their derivatives, and amino acids.

In one embodiment of the invention an elevated level of at least one, two, three or four of the biomarkers selected from the group consisting of sorbitol, alanine, myoinositol and cystathionine in the sample of the subject indicates the mitochondrial disorder and/or prognosis of said subject.

Sorbitol is a sugar alcohol, which may be synthesized via a glucose reduction reaction. Alanine is a non-essential amino acid, which is produced from pyruvate by transamination. Inositol is a sugar alcohol and myoinositol is one of its nine stereoisomers. Cystathionine is an intermediate in the synthesis of cysteine.

In one embodiment of the invention an increased level of at least one, two, three or four of the biomarkers selected from the group consisting of sorbitol, alanine, myoinositol and cystathionine in the sample of the subject indicates the mitochondrial disorder and/or prognosis of said subject. An increase of the level of a specific metabolite is preferably a significant increase.

In a further embodiment the levels of said four biomarkers in the sample of the subject are compared to the levels of said four biomarkers in a control sample or the levels of said four biomarkers in the sample of the subject are compared to the normal levels of said four biomarkers determined from a set of controls.

Only four biomarkers (sorbitol, alanine, myoinositol and cystathionine) need to be determined in the present invention. However, in one embodiment the method further comprises determining one or more further biomarkers in the sample of the subject, wherein one or more further biomarkers are selected from the group consisting of FGF21, GDF15, lactate and pyruvate and any combination thereof. In a very specific embodiment the method comprises determining at least biomarkers sorbitol, alanine, myoinositol, cystathionine, FGF21 and GDF15. FGF21 refers to fibroblast growth factor 21, which is the primary endogenous agonist of the FGF21 receptor. GDF15 refers to growth differential factor 15, which is a member of the transforming growth factor beta (TGF-β) superfamily, also secreted by the liver, especially in response to liver tissue injury. Lactate is a conjugate base of lactic acid, and L-lactate is constantly produced from pyruvate by lactate dehydrogenase (LDH) in a process of fermentation during normal metabolism and exercise. Pyruvate can be converted into carbohydrates via gluconeogenesis, to fatty acids or energy through acetyl-CoA, to the amino acid alanine and to ethanol.

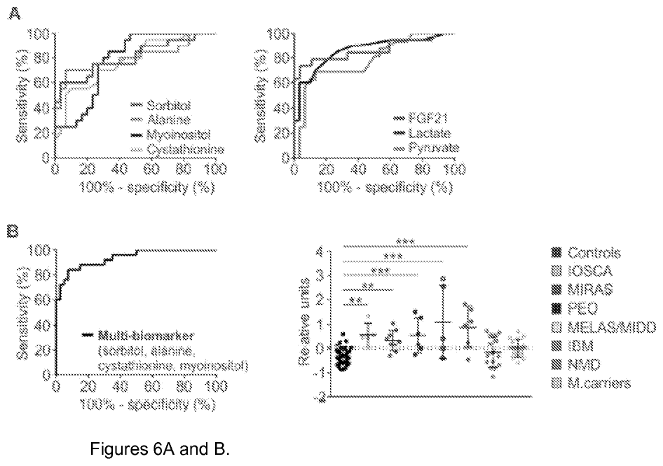

In a very specific embodiment of the invention ROC (receiver operating characteristic) curve i.e. a graphical plot is utilized for illustrating the diagnostic ability, i.e. the sensitivity and specificity, of the method or kit for detecting mitochondrial disorders by metabolites. ROC may be used when the method of the present invention is established. As an example, for each and every metabolite (e.g. in FIG. 6 there are sorbitol, myoinositol, cystathionine, alanine, FGF21, lactate and pyruvate), true positive rates (TPR or sensitivity) are plotted (e.g. using Graphpad program) in function of the false positive rate (FPR or 1-specificity) at various threshold settings, giving the curve graph for each metabolite and from there the area under the curve (AUC) is calculated. AUC reveals the chance that the result is correct. In the kit of the present invention, the absolute values of biomarkers in a sample are considered, e.g. against a control range.

In a specific embodiment of the invention TPR (or sensitivity) is calculated as follows: true positive number/(true positive number+false negative number), and/or specificity is calculated as follows: true negative number/(true negative number+false positive number), and/or

FPR is calculated as follows: 1—specificity.

True positive number=number of patients the metabolite classified as positive (disease)

False negative number=number of patients the metabolite classified as negative (healthy)

True negative number=number of controls (without the disease) the metabolite classified as negative (healthy)

False positive number=number of controls (without the disease) the metabolite classified as positive (disease)

In a very specific embodiment of the present invention a statistical program (e.g. GraphPad) or excel is utilized for calculating the diagnostic ability.

In one embodiment of the invention the mean centroid calculations used for ROC analysis are as follows: The original value of each of the four metabolites of each patient and control is taken and then the mean and standard deviation (SD)(of each metabolite) is calculated. Each metabolite original value is treated as follows: (original metabolite value−metabolite mean)/metabolite SD. This creates a new value (number) for each metabolite of every patient and control. Finally, the mean centroid value for each patient is calculated by calculating the mean of the new values (an average of the four metabolites' new values). Out of e.g. four original values per patient or control (if four metabolites) one mean centroid number is created. Overall (e.g. as shown in the FIG. 6), if all original values are low (like in controls) the calculated mean (seen in the figure) of the mean centroid values of controls is −1, if the half of the values are low and half are high (like in carriers) the mean of carriers is 0. If all values are high (like in patients) the mean is +1.

As used herein “significant” refers to statistically significant i.e. p≤0.05. Statistical methods suitable for the present invention are any common statistical methods known to a person skilled in the art. In a specific embodiment of the invention the statistical method for determining a decrease, increase, significant decrease or significant increase in the expression level includes but is not limited to a t-test, modified t-test, Shrinkage t-test, Fischer's exact test, one-way ANOVA and Dunnett's multiple comparison test. In a very specific embodiment of the invention a mean centroid for the four metabolites (sorbitol, alanine, cystathionine, myoinositol) is calculated for each and every subject as an overall predictive value for mitochondrial diseases and optionally tested with one-way ANOVA and Dunnett's multiple comparison test (e.g. all the patient groups and optionally carriers are compared to the control group).

Metabolites to be determined according to the present invention may be extracted from a sample with any extraction method known to a person skilled in the art, including but not limited to (cold) methanol extraction methods. For studying or analyzing levels of metabolites e.g. liquid chromatography and/or mass spectrometry (such as high resolution liquid chromatography-mass spectrometry (LC-HRMS)) may be utilized. In a very specific embodiment of the invention the method comprises protein precipitation with acetonitrile and formic acid and liquid chromatography with mass spectrometry. In one embodiment of the invention the study or analyses of metabolite levels is carried out in a plate format such as Ostro™ 96-well plate.

In some embodiments one or more control samples may be obtained from any control subject depending of the nature of the method. Optionally positive control samples showing increased biomarker levels compared to normal samples may be utilized in the present invention. Also, a quality control of the method may optionally be present in the method or within the kit of the present invention.

The kit of the present invention comprises at least tools for determining four biomarkers sorbitol, alanine, myoinositol and cystathionine from a sample of a subject, and optionally reagents (such as one or more selected from the group consisting of suitable extraction liquid(s) (e.g. acetonitrile, formic acid), reaction solutions, washing solutions, buffers and enzymes) for performing said test. In one embodiment “performing said test” refers to performing a test or method for determining at least four biomarkers sorbitol, alanine, myoinositol and cystathionine (e.g. the presence, absence, amount or concentration) in a sample of a subject. In one embodiment tools for determining the biomarkers may include one or more tools selected from the group consisting of probes enabling determination of the biomarkers, detection means, such as labels or colouring agents, enzyme(s) (such as an alanine converting enzyme, sorbitol dehydrogenase, phytase, and alkaline phosphatase), and one or more antibodies or antigen binding fragments specific for sorbitol, alanine, myoinositol and/or cystathionine (and optionally for one or more of FGF21, GDF15, lactate and/or pyruvate). The label(s) optionally utilized in the present invention can be any conventional labels, such as a radioactive label, an enzyme, a nucleotide sequence or a fluorescent compound. In general the presence, absence or amount of the biomarkers of the present invention in a sample can be detected by any suitable method known in the art. Therefore, the kit and tools may comprise any tools for carrying out the suitable detection methods including but not limited to enzymatic assays, immunological detection methods and combinations thereof.

In one embodiment, the method or kit of the present invention may comprise tools for an immunoassay comprising an antibody or an antigen binding fragment for the biomarkers of the present invention. The immunoassay can be either a competitive or non-competitive immunoassay. Competitive immunoassays include homogenous (e.g. fluorescence polarisation assay) and heterogenous (e.g. competitive ELISA) immunoassays. The immunoassay is not limited to but can be selected e.g. from the group consisting of ELISA, immunoPCR or FIA. In one embodiment the immunoassay may be for example a conventional sandwich test in microtiter wells or a lateral flow-test. Furthermore, any other assay types, such as agglutination test, lateral flow test, capillary electrophoresis, antibody arrays and/or microfluidic assay systems, or any combination thereof can be applied in the present invention. Indeed, the method or kit of the present invention may comprise use of one or more of said (immune)assays. In a specific embodiment the test kit comprises reagents for carrying out an (immune)assay. In a very specific embodiment of the invention, the method comprises an enzymatic assay and/or immunoassay (e.g. ELISA), or the kit comprises tools for an enzymatic assay and/or immunoassay such as an ELISA assay.

Detection mode of the method or immunoassay of the present invention can be any conventional detection mode including but not limited to colorimetric, fluorescent, paramagnetic, electrochemical or label free (e.g. surface plasmon resonance and quartz crystal microbalance) detection mode. Optionally determination may also comprise use of any suitable statistical methods known to a person skilled in the art.

In a specific embodiment of the invention, the kit is a plate-based kit or the method is carried out in a plate-based kit.

In one embodiment of the invention said kit is for the method of the present invention.

In a specific embodiment the kit comprises instructions for carrying out a method for determining at least four biomarkers sorbitol, alanine, myoinositol and cystathionine from a sample of a subject or for determining whether a subject has a mitochondrial disorder. E.g. said instructions may include instructions selected from the group consisting of instructions for carrying out the assay of the kit, instructions for extracting metabolites, instructions for separating metabolites with liquid chromatography (e.g. with ultra performance liquid chromatography), instructions for analyzing metabolites with mass spectrometry (e.g. triple quadruple mass spectrometry), instructions for interpreting the results, instructions for carrying out the statistical analysis and any combination of said instructions.

In a very specific embodiment of the invention the kit comprises tools to determine at least the four biomarkers sorbitol, alanine, myoinositol and cystathionine, reagents for performing said method, and optionally the reference levels (i.e. cut off levels) of suitable subjects, a concentration range determined from a group of normal healthy subjects for each biomarker, and/or instructions for carrying out a method for determining the biomarkers or determining whether a subject has a mitochondrial disorder.

In one embodiment the kit of the present invention further comprises tools for determining one or more biomarkers selected from the group consisting of FGF21, GDF15, lactate and pyruvate and any combination thereof. In a very specific embodiment the kit comprises tools for determining at least biomarkers sorbitol, alanine, myoinositol, cystathionine, FGF21, and GDF15. In one embodiment the kit comprises tools for determining the biomarkers by utilizing the same technique for all the biomarkers. In another embodiment the kit comprises tools for determining the biomarkers using different techniques for different biomarkers. For example, the kit may comprise tools for studying or analyzing levels of metabolites e.g. by liquid chromatography and/or mass spectrometry and/or immunoassay. Biomarkers comprising one or more of the following: sorbitol, alanine, myoinositol, cystathionine, FGF21, GDF15, lactate and/or pyruvate, can be determined with an immunoassay. Alternatively, e.g. sorbitol, alanine, myoinositol, cystathionine, lactate and/or pyruvate can be determined with liquid chromatography and/or mass spectrometry (such as LC-HRMS), and FGF21, GDF15 can be determined with an immunoassay (such as ELISA).

In one specific embodiment if in addition to an elevated level of one or more of sorbitol, alanine, myoinositol and cystathionine also both FGF21 and GDF15 are increased, a mitochondrial disorder (e.g. a defect affecting mitochondrial translation/mtDNA deletions) is very likely, e.g. the probability of a mitochondrial disorder being at least 90%, 91%, 92%, 93%, 94%, 95%, 96%, 97%, 98%, or 99%. In another specific embodiment an elevated level of GDF15 or FGF21 (e.g. increased or decreased) is associated with a specific disease. In a very specific embodiment an increased level of GDF15 without an increased level of FGF21, or vice versa is associated with IBM. Indeed, the present method and kit can be useful tools for both specific diagnoses as well as in differential diagnoses.

In a very specific embodiment of the present invention, when the sample to be used in the method or with tools of the kit is other than a sample obtained by a biopsy, the need of an invasive biopsy procedure for determining a mitochondrial disorder may be avoided.

In one embodiment of the invention the determination of at least four biomarkers is carried out in vitro. In another embodiment the kit of the present invention is for in vitro method. In vitro diagnostics refers to a medical and veterinary laboratory tests that are used to diagnose diseases and monitor the clinical status of patients using samples obtained from a subject.

The method or kit of the present invention is very sensitive and/or specific for mitochondrial diseases. The sensitivity and/or specificity can further be increased e.g. with biomarkers FGF21 and/or GDF15. In one embodiment of the invention the sensitivity of the method or kit of the present invention to find mitochondrial disease is more than 60%, more than 65%, more than 70%, more than 75%, more than 80%, more than 85%, more than 90% or more than 95%. In another embodiment of the invention the specificity of the method or kit of the present invention to find mitochondrial disease is more than 90%, more than 91%, more than 92%, more than 93%, more than 94%, more than 95%, more than 96%, more than 97%, more than 98%, or more than 99%. In a specific embodiment of the invention the sensitivity is more than 60%, 65%, 70%, 75%, 80%, 85%, 90% or 95% and/or the specificity is more than 70%, 75%, 80%, 85%, 90%, or 95%. In a very specific embodiment the sensitivity is more than 70% and/or the specificity is more than 90%.

A sample utilized in the present invention may be e.g. any organ, tissue, blood or cell sample. In one embodiment of the invention the sample is selected from the group consisting of a blood sample, plasma sample, serum sample, cheek tissue sample, urine sample, faeces sample, sputum sample, saliva sample, skin sample, muscle sample, cerebrospinal fluid, bone marrow, exhaled air sample, and any tissue or organ biopsy; most specifically the sample is a blood or muscle sample. Samples may be collected with any suitable method known to a person skilled in the art including but not limited to collecting blood, needle biopsy, aspiration, an open or closed biopsy or a biopsy obtained during a surgery (e.g. frozen sections). As an example, blood samples or other bodily fluids can be collected after an overnight fast. For blood, serum (e.g. no coagulant included) and/or plasma (e.g. with K2-EDTA) can be immediately separated from the peripheral venous blood e.g. by centrifugation. Alternatively, blood, e.g. peripheral venous blood, can be used as such for the present invention. In one embodiment the samples are frozen within few hours after the withdrawal and/or stored deep-frozen until analysis. Muscle samples (or any other biopsy samples) can be taken e.g. by needle biopsy, aspiration, conchotome or similar tool, by an open or closed biopsy, or a biopsy obtained during a surgery under local or general anesthesia. In one embodiment the muscle samples are snap frozen and/or stored deep-frozen until analysis.

In one embodiment of the invention a subject is a human (e.g. a child, an adolescent or an adult) or an animal (e.g. a mammal). A subject is in need of determining a mitochondrial disorder, predicting a prognosis of a mitochondrial disorder, or selecting or following up a treatment of a mitochondrial disorder.

Before classifying a subject as suitable for any method of the present invention, the clinician may for example study any symptoms or assay any disease markers of the subject. The clinician may suggest the method of the present invention for determining a mitochondrial disorder e.g. based on the results deviating from the normal or when having the suspicion of a mitochondrial disorder.

The tools and methods of the present invention can be utilized as first-line diagnostic tools in patients with symptoms of mitochondrial disorders (e.g. with muscle involvement). As an example, if levels of all or most of the biomarkers have increased (e.g. sorbitol, alanine, myoinositol and cystathionine and optionally FGF21 and/or GDF15), these patients would typically then be forwarded for muscle sampling, and next diagnostic examination could be next-generation sequencing analysis of a panel of mitochondrial disease genes, e.g. both nuclear and mtDNA. This approach speeds up the diagnostic rate of mitochondrial diseases, bring the diagnostic modalities also to primary care, as well as prioritizes patients for the invasive muscle biopsy procedure, minimizing the risk of complications.

In one embodiment of the present invention a specific treatment is selected for a subject having a mitochondrial disorder based on the marker profile in the sample of the subject. The method of the present invention based on determining specific biomarkers from a sample of a subject enables identifying subjects that are responsive/nonresponsive to a treatment of a mitochondrial disorder (e.g. including but not limited to treatment with vitamins, creatine, L-arginine, L-carnitine, coenzyme Q10, gene therapy, specific diet and/or physical therapy). An elevated level of at least one, two, three or four of the biomarkers sorbitol, alanine, myoinositol and cystathionine in the sample compared to the levels of said four biomarkers in a control sample enables the clinician to optimize the specific treatment. As used herein, the term “treatment” or “treating” refers to administration of at least one therapeutic agent to a subject for purposes which include not only complete cure but also amelioration or alleviation of disorders or symptoms related to a mitochondrial disorder in question. Therapeutically effective amount of an agent refers to an amount with which the harmful effects of a mitochondrial disorder are, at a minimum, ameliorated. The harmful effects of a mitochondrial disorder include but are not limited to one or more of the following: ptosis, external ophthalmoplegia, proximal myopathy and exercise intolerance, cardiomyopathy, sensorineural deafness, optic atrophy, pigmentary retinopathy, diabetes mellitus, fluctuating encephalopathy, seizures, dementia, migraine, stroke-like episodes, strokes, severe developmental delays, inability to walk, talk, see, or digest food, ataxia, spasticity, mid- and late pregnancy loss. The effects of therapeutic agents may be either short term or long term effects.

In one embodiment a method of treating a mitochondrial disorder comprises determining at least four biomarkers sorbitol, alanine, myoinositol and cystathionine from a sample of a subject and based on said results providing to the subject having a mitochondrial disorder or an increased risk for a mitochondrial disorder a treatment to prevent or retard said mitochondrial disorder. As an example, physical therapy or a diet may be sufficient treatment for a subject having an elevated or increased level of at least one biomarker from sorbitol, alanine, myoinositol and cystathionine in a sample. Pharmaceuticals or a combination of treatments could be utilized e.g. in cases wherein elevated or increased levels of at least one biomarker from sorbitol, alanine, myoinositol and cystathionine have been determined in a sample.

In one embodiment of the present invention a treatment of a subject having a mitochondrial disorder is followed up by determining at least the biomarkers sorbitol, alanine, myoinositol and cystathionine in a sample of the subject. In one embodiment the treatment has positive effects if a level of at least one, two, three or four of the biomarkers sorbitol, alanine, myoinositol and cystathionine in the sample of a patient having a mitochondrial disease decreases after or during said treatment. Said treatment has positive effects if an elevated level of at least one, two, three or four of the biomarkers sorbitol, alanine, myoinositol and cystathionine in the sample of a patient having a mitochondrial disease (e.g. when compared to sample/s from healthy individual/s or concentration range determined from a group of normal healthy subjects) decreases in concentration, towards the level of the healthy subjects concentration level/range after or during said treatment. On the other hand, when an elevated level of at least one, two, three or four of the biomarkers sorbitol, alanine, myoinositol and cystathionine in the sample of a patient having a mitochondrial disease (e.g. when compared to a sample from healthy individual/s or concentration range determined from a group of normal healthy subjects, and optionally before said treatment) does not change towards the level of the control sample, the treatment does not have positive effects. As used herein “positive effects” refers e.g. to complete cure or amelioration or alleviation of disorders or symptoms related to a mitochondrial disorder in question. Following up the combination of biomarkers of the present invention enables the clinician to optimize the treatment of a patient (e.g. to increase or decrease the dosage of a pharmaceutical or to change the pharmaceutical).

Follow-up of a subject after a treatment or when being under treatment can be carried out e.g. once a week, once every two or four weeks, or once, twice, three times, four times, or 5-12 times a year.

In one embodiment of the present invention a prognosis of a subject having a mitochondrial disorder is predicted based on the marker profile in the sample of the subject. An elevated level of at least one, two, three or four of the biomarkers sorbitol, alanine, myoinositol and cystathionine in the sample compared to the levels of said four biomarkers in a control sample enables the clinician to predict a prognosis. In one embodiment the prognosis is more positive if a level of e.g. one or two biomarkers is increased compared to a situation wherein a level of e.g. at least three or four biomarkers of the biomarkers sorbitol, alanine, myoinositol and cystathionine are increased in the sample of a patient. E.g. elevation or increase of one, two or three of the biomarkers sorbitol, alanine, myoinositol and cystathionine indicates a better prognosis compared to a situation wherein at least all four of said biomarkers are elevated or increased in a sample of a subject. In one embodiment a lack of elevation or increase of the specific biomarkers indicates a better prognosis compared to a situation wherein at least one, two, three or four of the biomarkers sorbitol, alanine, myoinositol and cystathionine are elevated or increased in a sample of a subject.

It will be obvious to a person skilled in the art that, as the technology advances, the inventive concept can be implemented in various ways. The invention and its embodiments are not limited to the examples described below but may vary within the scope of the claims.

### EXAMPLES

### Example 1

### Methods

The study was undertaken according to Helsinki Declaration, and approved by the ethical review board of Helsinki University Central Hospital (HUGH) with written and signed informed consents from the study subjects.

### Participants

Table 1 summarizes the patient data. We obtained plasma samples from nine MIRAS patients (OMIM #607459), and muscle biopsy samples from five of them. All patients were homozygous for the “MIRAS allele” (p.W748S+E1143G) in POLG, the nuclear gene encoding the catalytic subunit of the mitochondrial DNA polymerase gamma. MIRAS is an autosomal recessive disorder affecting mainly the central nervous system (CNS). The MIRAS patients in this study manifested typically with progressive gait disturbance, polyneuropathy, ataxia, and some with epilepsy, but signs of muscle pathology were absent of mild (respiratory deficient muscle fibers, mtDNA deletions and blood FGF21 concentration; Table 1; Hakonen et al. 2005; Lehtonen et al. 2016). We also collected plasma from 16 non-manifesting MIRAS family members heterozygous for the MIRAS allele (“MIRAS carriers”). The MELAS (OMIM #540000)/MIDD (maternally inherited diabetes and deafness; OMIM #520000) patients carried a heteroplasmic m.3243A>G point mutation in mtDNA tRNALeu(UUR) gene (Goto, Nonaka, and Horai 1990). Plasma samples were obtained from five MELAS patients and muscle samples from two patients. The patients manifested in the late adulthood (˜40 years of age) with different combinations of mitochondrial myopathy and ragged-red fibers (RRFs), cardiomyopathy, diabetes mellitus, hearing loss and stroke-like episodes. MELAS patients showed high amount of respiratory chain deficient fibers in their muscle, and were heteroplasmic for the mutant mtDNA in the skeletal muscle (range 50-90%) and urine epithelial cells (65-80%) as determined by minisequencing (Suomalainen et al. 1993). They also showed high FGF21 concentration in their blood (Table 1; the patients were described in Lehtonen et al. 2016). Additionally, we utilized six serum samples from patients with inclusion body myositis (IBM; OMIM #147421). IBM is typically a sporadic muscle disease characterized by progressive weakness and wasting of distal muscles, the muscle samples show inflammation and typical findings of mitochondrial myopathy—a high amount of respiratory chain deficient muscle fibers—but normal level of blood FGF21 (Table 1; Suomalainen et al. 2011; Lehtonen et al. 2016). We therefore consider IBM a secondary mitochondrial disease, included in the present disease target group. As “non-mitochondrial disease controls” we analyzed serum metabolomes from 15 patients with different neuromuscular disorders (NMD; Suomalainen et al. 2011; Lehtonen et al. 2016): Becker's muscle dystrophy (DMD), myotonic dystrophy type I (DMPK) and II (ZNF9), motoneuron disease (unknown), muscle weakness (CAPN3), oculopharyngeal muscular dystrophy (PABPN1), late-onset Pompe's disease (GAA), spinal muscular atrophy type II (SMN1) and III (unknown), and Welander's muscular dystrophy (TIA1; Table 1). To compare the parallel disease specific signatures of all available genetically defined mitochondrial disease groups, we also re-analyze metabolomic data from blood and muscle of patients with progressive external ophthalmoplegia (PEO) and infantile-onset spinocerebellar ataxia (IOSCA; Nikkanen et al. 2016). The PEO cohort included patients with autosomal dominant PEO with TWNK mutations (TWNK-PEO, OMIM #609286; Spelbrink et al. 2001), a patient with recessive mutations in POLG (POLG-PEO, OMIM #157640; Luoma et al. 2004) and patients with a sporadic single heteroplasmic large mtDNA deletion (Del-PEO; Table 1). Muscle samples were obtained from three TWNK-PEO patients and two Del-PEO patients. IOSCA (OMIM #271245) is caused by a homozygous recessive mutation in TWNK (Nikali et al. 2005). Blood samples were obtained from 30 healthy volunteers (median age 42 years) and muscle samples from 10 healthy volunteers (median age 48.5 years).

TABLE 1
Characteristics of mitochondrial and non-mitochondrial neuromuscular disease patients

| | IOSCA | MIRAS | PEO | MELAS/MIDD | IBM | NMD |
|---|---|---|---|---|---|---|
| | N = 5* | N = 9 | N = 8 | N = 5 | N = 6 | N = 15 |
| Gender (n) | 2F, 3M | 2F, 7M | 3F, 5M | 2F, 3M | 3F, 3M | 12F, 3M |
| Age of | 1-2 | 29.6 | 27.8 | 39.3 | 61.4 | 26.3 |
| onset | | (18.0-44.0) | (21.0-35.0) | (30.0-48.0) | (49.0-83.0) | (2.0-60.0) |
| (years)^{a} | | | | | | |
| Age at | 38.6 | 41.2 | 50.0 | 54.0 | 71.0 | 49.9 |
| sampling | (33.0-42.0) | (21-52.0) | (39.0-57.0) | (39.0-68.0) | (58.0-85.0) | (23.0-77.0) |
| (years)^{a} | | | | | | |
| FGF21 | — | 132.5 | 454.0 | 562.0 | 57.0 | 114.0 |
| (pg/ml)^{b, c} | | (51.0-279.8) | (222.0-604.3)† | (188.5-2569.0)‡ | (34.3-287.8) | (24.0-190.0) |
| Inheritance | AR, TWNK | AR, POLG | AD, TWNK | Maternal, | Sporadic | AD or AR, |
| disease | p. Y508C | p. W748S + | 13AA dup | mtDNA | | DMPK, ZNF9, |
| gene, | | E1143G | (TWNK-PEO); | m.3243A > G, | | CAPN3, |
| amino acid | | | AR, POLG | tRNA^{Leu(UUR)} | | PABPN1, |
| change | | | p. A1105T/N468D | | | SMN1, TIA1 |
| | | | (POLG-PEO); | | | or unknown |
| | | | Sporadic, | | | |
| | | | mtDNA single | | | |
| | | | deletion | | | |
| | | | (Del-PEO) | | | |
| Histological | None | 1-5% | POLG/TWN | 5-30% | 1-8% | Dystrophy, |
| findings | | COX−/SDH+ | K-PEO: 5-12% | COX−/SDH+ | COX−/SDH+ | hypertrophy, |
| in skeletal | | fibers | COX−/SDH+; | fibers | fibers | normal |
| muscle | | | Del-PEO: 30-60% | | | respiratory |
| | | | COX−/SDH+ | | | chain |
| MtDNA | MtDNA | MtDNA | Heteroplasmic | Heteroplasmy; ~70% | multiple | none |
| consequences | depletion | depletion, | multiple mtDNA | of mutant mtDNA | mtDNA | |
| | in brain | small amount | deletions, or | in muscle | deletions | |
| | and liver | of heteroplasmic | single large | and urine | | |
| | | multiple mtDNA | mtDNA deletion | epithelial | | |
| | | deletions in | in skeletal | cells | | |
| | | skeletal muscle | muscle | | | |
| Muscle | − | −/+ | + | ++ | ++ | ++ |
| symptoms | | | | | | |
| Clinical | Childhood- | Ataxia, | Mitochondrial | Mitochondrial | Distal | |
| symptoms | onset ataxia, | neuropathy, | myopathy, | myopathy, | progressive | |
| | neuropathy, | epilepsy, | ptosis, | cardiomyopathy, | muscle | |
| | athetosis, | psychiatric | progressive | diabetes | weakness | |
| | hearing loss, | symptoms, | external | mellitus, | | |
| | epilepsy, | cognitive | ophthalmoplegia, | hearing loss, | | |
| | hepatopathy | decline, | exercise | stroke-like | | |
| | | obesity/insulin | intolerance | episodes | | |
| | | resistance | | | | |

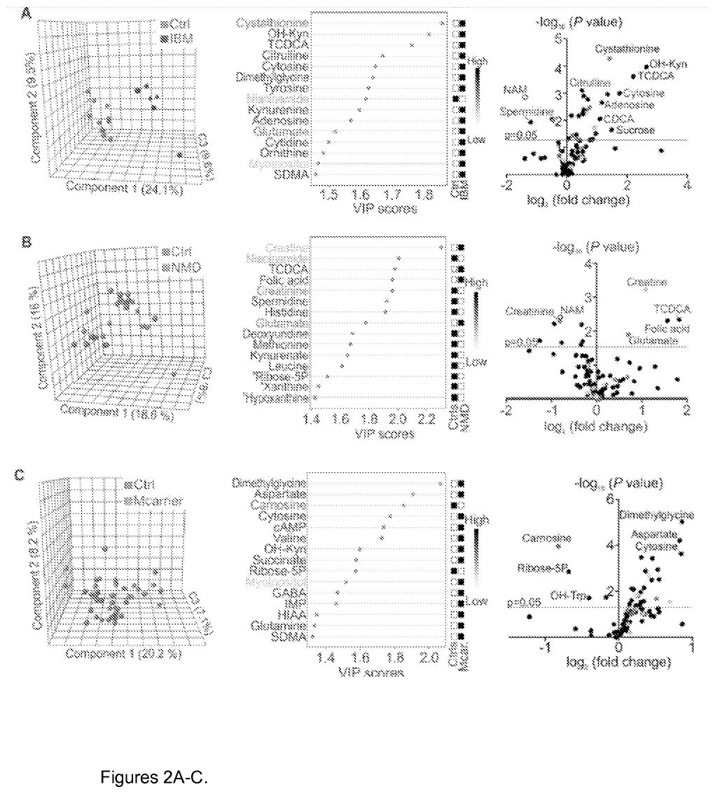

^{a}Values represent mean with minimal and maximal age.
^{b}Values represent median with interquartile range.
^{c}Normal value for FGF21 ≤ 331 pg/ml (Lehtonen et al. 2016).
‡P = 0.009,
†P = 0.002 (non-parametric Kruskal-Wallis test).
*Additional IOSCA child patient (four years of age; FIG. 2B). This child patient, however, was not included in the overall statistical analysis due to lack of appropriate age- and gender-matched control samples.
−, muscle phenotype not present; +, mild muscle pheno-type; ++, primary muscle phenotype. AR, autosomal recessive; AD, autosomal dominant; COX−/SDH+, cytochrome C oxidase-negative/succinate dehydrogenase-positive fibers; F, female; M, male; n, number; mtDNA, mitochondrial DNA.

### Blood and Muscle Samples

Blood samples were taken after an overnight fasting during an outpatient visit at Helsinki University Hospital. Serum (no coagulant included) and plasma (with K2-EDTA) were immediately separated from the peripheral venous blood by centrifugation at 3000 g at +4° C. for 15 minutes and stored at −80° C. until analysis. Muscle samples were taken by needle biopsy from vastus lateralis muscle under local anaesthesia, snap frozen and stored at −80° C. until analysis.

### Targeted Metabolomics Analysis

Serum/plasma and muscle metabolites were extracted and analysed as previously described (Khan et al., 2014; Kolho et al., 2017; Nandania et al. 2018; Nikkanen et al. 2016). Briefly, metabolites were extracted from frozen muscle samples (10-35 mg) homogenized with extraction solvent (1:30, sample:solvent) and 100 μl of serum/plasma samples (1:4, sample:solvent), separated with Waters Acquity ultra performance liquid chromatography and analysed with triple quadruple mass spectrometry. Complete method description and instrument parameters, including thorough validation of the analytical method according to European Medical Agency guidelines, is reported separately (Nandania et al. 2018). In blood, 94 metabolites were measured. However, at the time when we performed the muscle metabolite analysis, our metabolite set was updated to 111, including methionine intermediates and acylcarnitines.

### Statistical Analysis

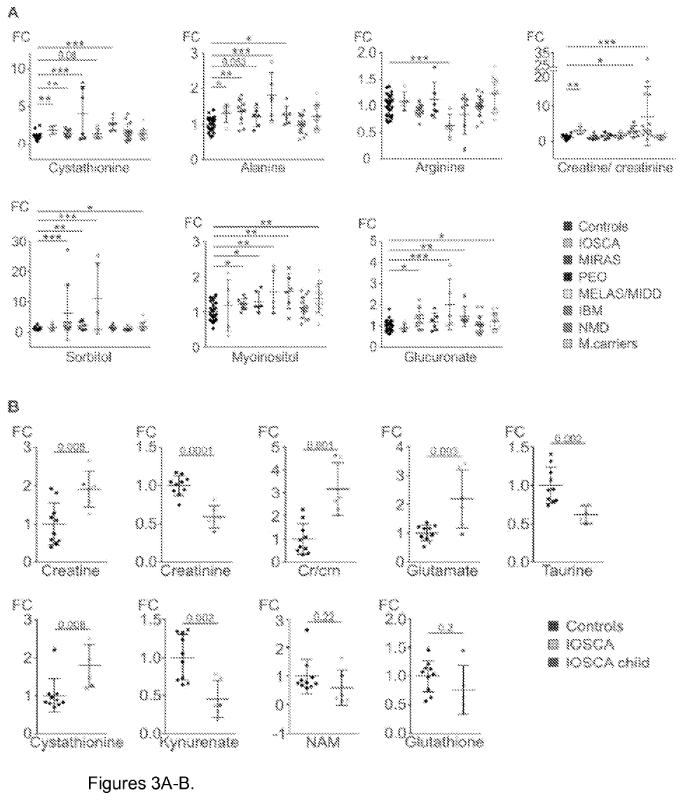

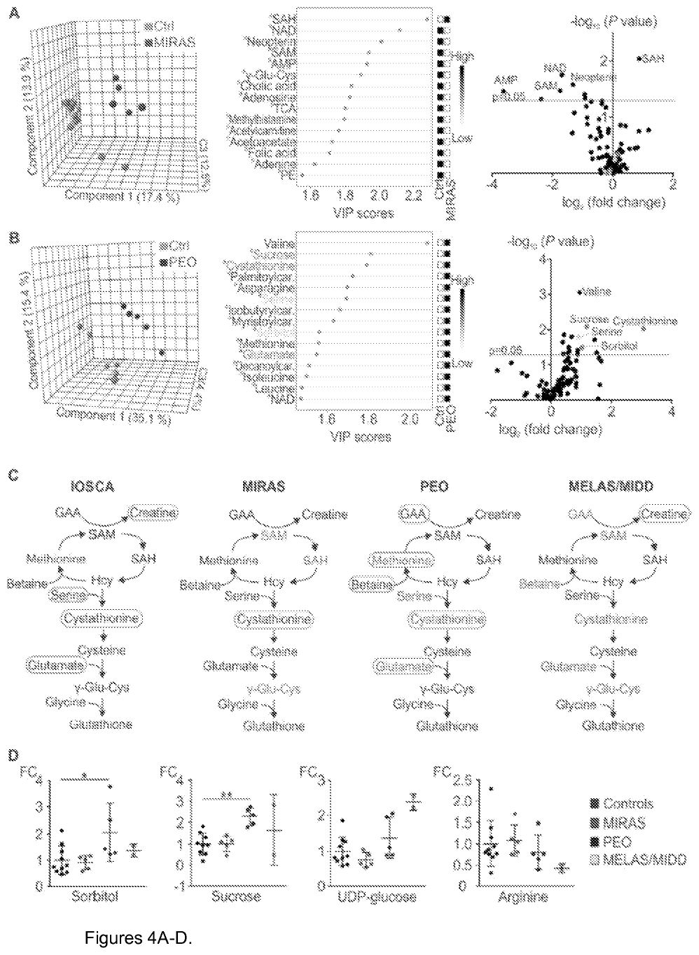

Targeted metabolomics data was analysed by using MetaboAnalyst 3.0 (www.metabolanalyst.ca; Xia et al. 2015, 2009). The data were log-transformed and autoscaled before statistical analysis. Plasma metabolomes of MIRAS (n=9), PEO (n=6), MELAS (n=5) and MIRAS carriers (n=16) were compared to plasma of controls (n=30). Serum metabolomes of IOSCA (n=5), IBM (n=5) and NMD (n=15) patients was compared to serum of controls (n=10). Individual metabolite values are shown for the one additional IOSCA child patient (FIG. 3B), to show the relevance of IOSCA findings in early vs late-stage disease. However, this child patient was not included in the overall statistical analysis of adult IOSCA patients due to lack of appropriate age- and gender-matched control samples. Muscle metabolomes of MIRAS (n=5) and PEO (n=5) patients were compared to muscle of controls (n=10 and n=7, respectively). Differences among controls and patient groups were tested with univariate analysis, two sample T-test. Metabolites were tested for false positivity (FDR) with Benjamini-Hochberg method with a critical value of 0.2. For multivariate regression, we performed partial least squares-discriminant analysis (PLS-DA) with variable importance in projection (VIP). The cross-validation of PLS-DA model was done with leave-one-out cross-validation (LOOCV) method (MetaboAnalyst 3.0). Due to the small amount of female MIRAS and PEO patients, we tested the effect of gender on blood metabolome among our controls (females n=16, males n=14). No metabolites with FDR<0.2 were significantly changed between male and female controls, therefore we included all male and female controls in MIRAS and PEO blood analysis (all figures). Due to small amount of MELAS muscle samples (n=2), statistical analysis was not possible (FIG. 4). Global test was used for the pathway enrichment analysis, and relative-betweeness centrality method was used for pathway topology analysis (MetaboAnalyst 3.0). Sensitivity and specificity were determined by the univariate ROC analysis, and AUC was determined (GraphPad PRISM 6; GraphPad software, La Jolla, Calif.). A mean centroid for metabolites with the highest AUC (cystathionine, alanine, sorbitol and myoinositol) was calculated for each patient as an overall predictive value and tested with one-way ANOVA and Dunnett's multiple comparison test (GraphPad PRISM 6). The mean centroid values of the four-metabolite biomarker of controls, IOSCA, MIRAS, PEO and MELAS were used for sensitivity and specificity determination by ROC curve, and AUC was calculated (GraphPad PRISM 6). Creatine/creatinine ratio between controls and patients was tested with Mann-Whitney test (GraphPad PRISM 6).

### Results

### Metabolomic Analysis of Blood Reveals Disease-Specific Biomarker Profiles

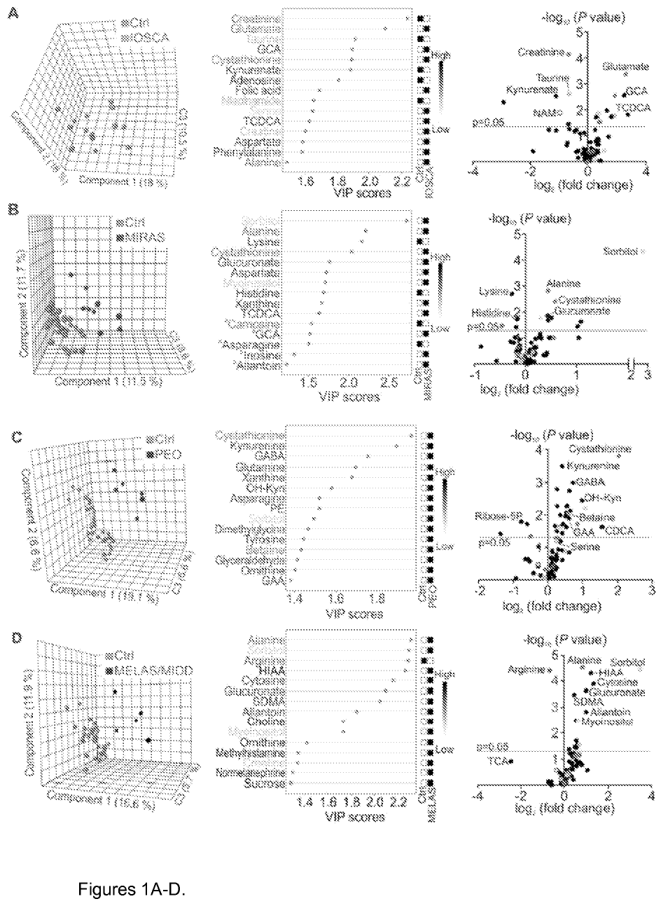

We performed high-throughput targeted semiquantitative analysis of 94 metabolites in blood samples of patients with mtDNA maintenance disorders (IOSCA; mitochondrial recessive ataxia syndrome, [MIRAS]; progressive external ophthalmoplegia/mitochondrial myopathy, [PEO]), or defect of mitochondrial translation (mitochondrial myopathy, encephalomyopathy, lactic acidosis and stroke-like episodes [MELAS]/maternal-inherited diabetes and deafness, [MIDD]); as well as of IBM patients, MIRAS carriers and non-mitochondrial neuromuscular disorder (NMD) patients (Table 1). The patient and control groups were analysed by the partial least squares discriminant analysis (PLS-DA; FIGS. 1 and 2). Metabolites with the highest separation power in PLS-DA were ranked by variable importance in projection (VIP) scores (FIGS. 1 and 2), described below for each disease.

IOSCA blood metabolome clustered separate from the controls (FIG. 1A). The metabolic profile of this epileptic encephalohepatopathy showed a strong component of creatine, bile acid and transsulfuration pathway changes. Low amounts of creatinine (fold change [FC]−1.6, P<0.001), the secreted breakdown product of creatine, suggested increased creatine turnover, which was also supported by significantly increased creatine/creatinine ratio (FIG. 3A), despite the increased amount of creatine in the blood (FC+1.9, P=0.017). Decreased steady-state kynurenate (FC−2.2, P=0.003) and niacinamide (NAM; FC−2.0, P=0.013; FIG. 1A) pointed to altered NAD⁺ synthesis pathway and an increase in NAD⁺ demand. The serine-driven transsulfuration pathway (Nikkanen et al. 2016) imbalance was marked by increased upstream metabolites serine (FC+1.3, P=0.013), glutamate (FC+2.4, P<0.001) and cystathionine (FC+1.9, P=0.003), but depletion of transsulfuration-dependent taurine (FC−1.6, P=0.002) and reduced form of glutathione (FC−2.2, P=0.063). Two bile acids, glycocholic acid (GCA; FC+2.4, P=0.003) and taurine-conjugated taurochenodeoxycholic acid (TCDCA; FC+2.6, P=0.015) were increased (FIG. 1A). Glutathione depletion indicates decreased potential for antioxidant capacity in IOSCA. Additionally, we analyzed a blood sample of an IOSCA child patient (four years of age) who showed high creatine/creatinine ratio and low taurine and kynurenate (FIG. 3B). The significant depletion of kynurenate and the significant depletion of niacinamide are both consistent with depletion of NAD⁺.

MIRAS blood metabolome, clustered separately from controls. The patients showed significant increase of carbohydrate derivatives, i.e. sorbitol (FC+6.2, P<0.0001), glucuronate (FC+1.4, P=0.014) and myoinositol (FC+1.2, P=0.017; FIG. 1B, 3A). Other changes included increased alanine (FC+1.4, P=0.002) and decreased lysine (FC−1.2, P=0.002) and carnosine (FC−1.4, P=0.034), involved e.g. in inactivation of methylglyoxal, a product of high sugars. Similar to IOSCA, cystathionine was increased (FC+1.5, P=0.004; FIG. 1B), whereas other transsulfuration or creatine metabolites were not significantly changed (FIG. 3A).

In PEO patients, the blood metabolome clustered separately from controls (FIG. 1C). The significantly changed metabolites included elevated cystathionine (FC+4.1, P<0.001), phosphoethanolamine (PE; FC+1.5, P<0.005), glutamine (FC+1.2, P=0.002) and sorbitol (FC+2.1, P=0.006; FIG. 1C, 3A). Overall, PEO blood showed a wide upregulation of amino acids and purine precursors (xanthine and xanthosine; both FC+1.5) as previously reported (Nikkanen et al. 2016; Ahola et al. 2016), and an increase in NAD+ synthesis pathway (kynurenine [FC+1.3, P<0.001]; 3-hydroxy-DL-kynurenine [FC+2.0, P=0.004]; FIG. 1C). Furthermore, unmethylated metabolite precursor of creatine, guanidinoacetic acid (GAA; FC+1.5, P=0.012; FIG. 1C), was increased suggesting deficient metabolite methylation.

The MELAS/MIDD blood metabolome clustered separately from the controls (FIG. 1D). The results showed remarkably increased carbohydrate derivatives: sorbitol (FC−F11.1, P<0.001), glucuronate (FC+2.0, P<0.001), myoinositol (FC+1.6, P=0.003; FIG. 3A) and sucrose (FC+1.5, P=0.035). Amino acids were in general higher than controls, including alanine (FC+1.8, P<0.0001), with an exception of significantly decreased arginine (FC−1.6, P<0.0001; FIG. 3A), which was specific for MELAS/MIDD in our material. These changes were not explained by diabetes, as they remained MELAS/MIDD-specific even when we compared all patients with increased insulin resistance to normoglycemic patients.

We then asked whether inclusion body myositis, a sporadic inflammatory muscle disease with secondary findings of mitochondrial myopathy in the muscle (respiratory chain deficient muscle fibres, multiple mtDNA deletions), would share blood metabolic features with primary respiratory chain deficiencies. IBM blood metabolome clustered separately from controls (FIG. 2A). The IBM metabolic profile was defined by elevated cystathionine (FC+2.7, P<0.0001), dimethylglycine (FC+2.6, P=0.001), TCDCA (FC+4.6, P<0.001) and citrulline (FC+1.4; P<0.001). The alternative NAD⁺ synthesis pathway was highly upregulated: kynurenine (FC+1.6, P=0.002) and its hydroxylated form, 3-hydroxy-DL-kynurenine (FC+6.2, P<0.001), were significantly induced, however, niacinamide was reduced (FC−2.6; P=0.001; FIG. 2A). Furthermore, IBM showed significant increases of nucleotide synthesis precursors (e.g. adenosine, deoxycytidine, cytidine and cytosine; FC+2.2, −1.3, +1.5, +3.4, respectively), as well as carbohydrate derivatives: sucrose, myoinositol and glucuronate (FC+2.8, +1.6 and +1.5, respectively; FIG. 2A, 3A). Also creatine/creatinine ratio was significantly increased, suggesting low creatine pools (FIG. 3A). Overall, IBM blood metabolome resembled the respiratory chain deficiencies (IBM shared 39% significantly changed metabolites with PEO) rather than NMDs (IBM and NDMs shared 23% of significantly changed metabolites).

To define which changes were specific for mitochondrial diseases and which were general consequences of muscle disease, we analyzed a group of heterogeneous non-mitochondrial neuromuscular diseases. The PLS-DA model of NMD patients clustered separately from controls (FIG. 2B). The NMD metabolome was discriminated by creatine metabolism (creatine FC+2.1, P<0.001; creatinine FC −1.8, P=0.005) with increased creatine/creatinine ratio (FIG. 3A), supporting depleted creatine pool. Furthermore, TCDCA (FC+3.6, P=0.005), folic acid (FC+3.0, P=0.005) and glutamate (FC+1.6, P=0.012) were increased and niacinamide (FC−1.7, P=0.004), spermidine (FC−1.9, P=0.006) and histidine (FC−1.2, P=0.006) were reduced (FIG. 2B). However, cystathionine and alanine, increased in both primary (IOSCA, MIRAS, PEO and MELAS) and secondary (IBM) mitochondrial disease patients, were not elevated in NMD patients, or in healthy controls or MIRAS carriers (FIG. 3A).

The blood metabolome of the heterozygous carriers of the recessive MIRASallele showed separation from controls (FIG. 2C). Similar to MIRAS patients, they had increased glucuronate (FC+1.2, P=0.029), sorbitol (FC+1.6, P=0.029) and myoinositol (FC+1.3, P=0.002; FIG. 3A) and low carnosine (FC−1.8, P<0.001). Cystathionine and alanine, the strongest markers of MIRAS, however, were not increased (FIG. 3A). In general, the metabolic profile of MIRAS carriers revealed subtle but significant changes, including dimethylglycine (FC+1.8, P<0.0001), aspartate (FC+1.8, P<0.0001) and cytosine (FC+1.8, P<0.001; FIG. 2C). The results suggest that carrier status of one MIRAS allele is not completely neutral for metabolism.

Methylation Cycle and Glutathione Pathway are Affected in Muscle of Mitochondrial Disease Patients

In order to compare the blood metabolomic findings with the primarily affected tissue to understand the tissue-specific changes, we performed targeted semiquantitative analysis of 111 metabolites in muscle from patients and control subjects. MIRAS is primarily a nervous system disorder, however, the patients carry a small amount of multiple mtDNA deletions in their skeletal muscle (Table 1; Hakonen et al. 2008), similar to PEO patients. MIRAS muscle metabolome was separated from controls in PLS-DA (FIG. 4A). The most significantly changed metabolites (false discovery rate [FDR]<0.5) were the major methyl carriers, elevated S-Adenosyl-L-homocysteine (SAH; FC+1.8, P=0.009) and reduced S-Adenosyl-L-Methionine (SAM; FC—3.4, P=0.034; FIG. 4A). This was an indication for methyl cycle imbalance in MIRAS muscle. However, the MIRAS muscle metabolite signature did not overlap with the blood biomarker profile (FIG. 4C), e.g. low carbohydrate derivatives in muscle (FIG. 4D), suggesting that the metabolic changes in the blood likely reflected metabolism of another affected tissue, such as the brain or liver; indeed, muscle manifestation in MIRAS is mild or completely lacking.

The PEO and MELAS/MIDD patients in this study had mainly muscle/cardiac symptoms. The PEO muscle metabolites separated from controls in PLS-DA model (FIG. 4B), and the muscle metabolic profile revealed changes in key metabolites of the methyl cycle and glutathione metabolism: cystathionine was remarkably increased (FC+8.3, P=0.009), and methionine (FC+1.5, P=0.032) and serine (FC+1.9, P=0.016; FIG. 4B) were elevated (FDR<0.3). The carbohydrate metabolites were also prominent in PEO muscle (FIG. 4D). These metabolites overlapped well with the metabolites changed in PEO blood (FIG. 4C, D; Nikkanen et al. 2016). Similar to PEO, cystathionine was increased in MELAS/MIDD muscle (FC+1.3) as were other contributors to the transsulfuration cycle, namely gamma-glutamyl-cysteine (γ-Glu-Cys; FC+1.4), SAM (FC+1.9) and glutamate (FC+1.2). In contrast, adenosine (FC−3.6), GAA (FC−3.4) and betaine (FC−2.4) were reduced. The metabolites from MELAS/MIDD blood and muscle partially overlapped (FIG. 4C, D): e.g. low arginine (blood [FC−1.6; FIG. 3A] and muscle [FC−2.4; FIG. 4D]). The findings of PEO and MELAS/MIDD patients support the conclusion that the blood metabolome reflects at least partially the metabolome of the diseased affected tissue.

Pathway Analysis: One-Carbon Metabolism Remodeled in mtDNA Maintenance Disorders

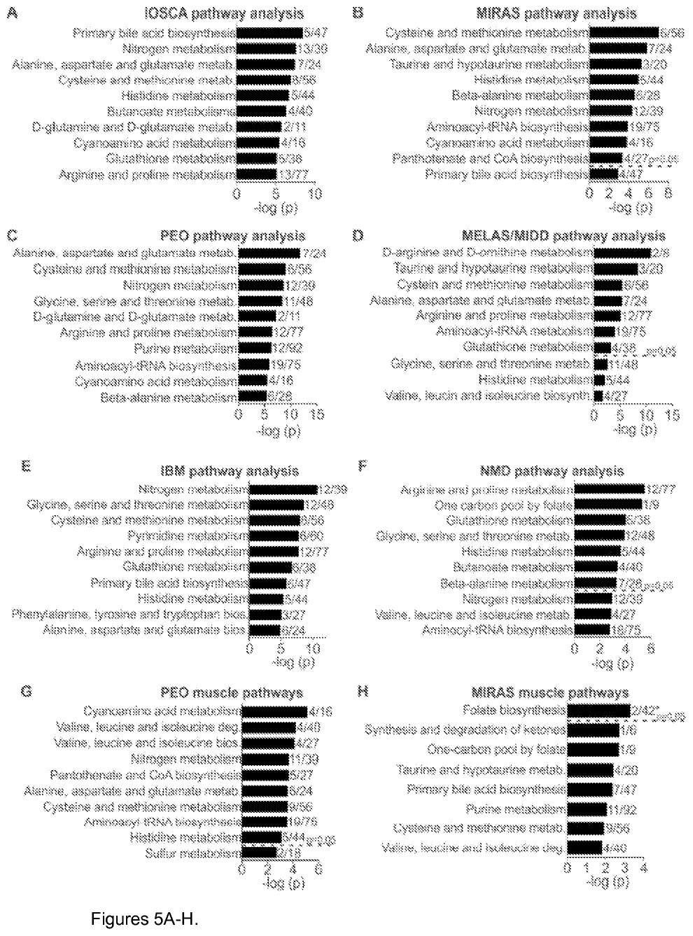

Pathway analysis of the full metabolomes of blood showed several significantly changed pathways common to all mitochondrial disorders, but not to NMDs (FIG. 5 shows top 10 pathways with 10% metabolites detected in the pathway). Transsulfuration pathway (cysteine and methionine metabolism) and amino acid biosynthesis pathway (alanine, aspartate and glutamate biosynthesis) were aberrant in mtDNA expression disorders (mtDNA maintenance/translation: IOSCA, MIRAS, PEO, MELAS) and IBM (FIG. 5A-E), as well as in muscle of PEO patients (FIG. 5G); the cysteine and methionine metabolism being among the top four significant pathways in blood, and folate metabolism being also prominent in MIRAS muscle (FIG. 5H). Transsulfuration pathway or the amino acid biosynthesis pathway were not significantly changed in NMD patients (FIG. 5F). Purine/pyrimidine synthesis was common to muscle manifesting disorders including NMD (FIG. 5C-F).

Sorbitol, Myoinositol, Alanine and Cystathionine: A Multi-Biomarker for Mitochondrial Disorders

We then tested the performance of metabolites as disease biomarkers in a pooled set of mitochondrial disease patients (n=20), asking which metabolites would have the best sensitivity and specificity for mitochondrial disease to distinguish them from healthy controls (n=30). By receiver operating characteristic (ROC) curve analysis, the top four significant metabolites with the highest area under curve (AUC) were sorbitol 0.81 (95% confidence interval [CI] 0.68-0.94, P=0.0003), alanine 0.81 (95% CI 0.67-0.94, P=0.0003), myoinositol 0.79 (95% CI 0.66-0.91, P=0.0007) and cystathionine 0.78 (95% CI 0.65-0.91, P=0.001; FIG. 6A), which we together call “multi-biomarker” for mitochondrial disorders. We then compared it to conventional blood biomarkers: fibroblast growth factor 21 (FGF21), a serum biomarker of muscle-manifesting mitochondrial disorders, lactate and pyruvate. For the same set of patients and controls, FGF21 had the highest AUC 0.87 (95% CI 0.74-0.99, P=0.0001), followed by lactate 0.86 (95% CI 0.76-0.97, P=0.0001) and pyruvate 0.78 (95% CI 0.64-0.93, P=0.0017; FIG. 6A). We then compared sensitivity of the four metabolites and the conventional blood biomarkers to find a mitochondrial disorder. FGF21 showed the highest sensitivity of all (68%; 95% CI 43.5-87.4;

FIG. 6A), when including all mitochondrial disorder patients, and when considering only muscle manifesting mitochondrial disorders—known to induce FGF21 secretion—its sensitivity in this material was 91% (95% CI 66.4-100.0). Lactate and pyruvate showed sensitivity 45% (95% CI 23.1-68.5) and 13% (95% CI 1.6-38.4), respectively, and specificity 97% (95% CI 82.8-99.9). As single metabolites, sorbitol and alanine showed sensitivity of 55% (95% CI 31.5-76.9) and specificity 97% (95% CI 82.8-99.9), and for myoinositol and cystathionine the sensitivity was 25% (95% CI 8.7-49.1), and specificity 93.3% (95% CI 77.9-99.2) and 97% (95% CI 82.8-99.9), respectively (FIG. 6A), to identify mitochondrial disorders. However, when we combined the four metabolites together and calculated mean centroid values from sorbitol, alanine, cystathionine and myoinositol for all patients and controls, the primary and secondary mitochondrial disorders differed significantly from controls, MIRAS carriers and NMDs (FIG. 6B). The sensitivity of this blood multi-biomarker to find mitochondrial disorder raised to 76% (95% CI 54.9-90.6) and specificity to 95% (95% CI 83.1-99.4) with AUC 0.94 (95% CI 0.88-0.995, P=0.0001; FIG. 6B).

### Example 2

Serum Biomarker Analyses for FGF21 and GDF15:

The serum samples were snap-frozen and stored at −80° C. before analysis. The biomarkers were analyzed with commercially available kits (FGF21: Biovendor, Brno, Czech Republic; the results exceeding the linear range were replicated with the kit of R&D Systems, Minneapolis, Minn. GDF15: R&D Systems) according to the manufacturers' instructions. The plate absorbances were measured using a SpectraMax 190 absorbance microtiter plate reader (Molecular Devices, Sunnyvale, Calif.).

Statistical Analyses:

If the causative mutation involved a protein known to be associated with mitochondrial function and was present in a database (https://mseqdr.org/), the disease was considered to be a mitochondrial disease. The odds ratios were calculated using Fisher's exact test. Association of FGF21 values to GDF15 values was done using Spearman's rank correlation analysis. Association was considered significant if the r-value exceeded 0.5 and two-sided p-value was <0.05. In this case, a linear regression model was performed and the R² and P-values for goodness of fit are reported. All statistical analyses were performed by PRISM 7.0 (Graph Pad software, La Jolla, Calif.).

Results:

We confirmed in this material that muscle manifestation and defect of mtDNA expression system (translation, mtDNA deletions) induced both markers. The odds ratio (OR) for having a mtDNA expression disorder was 42 (n=23, CI 3.17-556.5, p<0.01), if one of the biomarkers showed intermediate or high concentrations in a muscle manifesting disorder. If one of the biomarkers were induced (intermediate or pathological) in a patient with a myopathic disease, an mtDNA expression disease was the cause in 93% likelihood (CI 0.68-0.99, p<0.01, positive predictive value). Patients, who did not have a specific diagnosis and showed pathological biomarker values, also showed more symptoms and findings suggestive of mitochondrial myopathy (PEO, COX deficient fibers, OXPHOS defect) than those, with at least one of the biomarkers in normal range.

### Example 3

The samples of subjects suspected to have a mitochondrial disorder are collected as described in example 1. The levels of biomarkers sorbitol, alanine, myoinositol and cystathionine are determined as described in example 1. Furthermore, one or more biomarkers selected from the group consisting of FGF21, GDF15, lactate, pyruvate, and any combination thereof can be determined from the samples of the subjects (see e.g. example 2).

Patients with an increased level of at least the biomarkers sorbitol, alanine, myoinositol and cystathionine and diagnosed with a mitochondrial disorder are treated with a suitable pharmaceutical for a mitochondrial disorder. The levels of the four biomarkers and optionally one or more from the group consisting of FGF21, GDF15, lactate, pyruvate, and any combination thereof, are followed up after and/or under the treatment period by determining the levels of said biomarkers e.g. as described in examples 1 and 2. Lactate and/or pyruvate can be determined e.g. by an immunoassay (e.g. ELISA).

The treatment has positive effects if an elevated level of at least one, two, three or four of the biomarkers sorbitol, alanine, myoinositol and cystathionine (and optionally one or more from the group consisting of FGF21, GDF15, lactate, pyruvate, and any combination thereof) in the sample of a patient having a mitochondrial disease (e.g. when compared to sample/s from healthy individual/s or concentration range determined from a group of normal healthy subjects) decreases in concentration, towards the level of the healthy subjects concentration level/range after or during said treatment. On the other hand, when an elevated level of at least one, two, three or four of the biomarkers sorbitol, alanine, myoinositol and cystathionine (and optionally one or more from the group consisting of FGF21, GDF15, lactate, pyruvate, and any combination thereof) in the sample of a patient having a mitochondrial disease (e.g. when compared to a sample from healthy individual/s or concentration range determined from a group of normal healthy subjects, and optionally before said treatment) does not change towards the level of the control sample, the treatment does not have positive effects. As used herein “positive effects” refers e.g. to complete cure or amelioration or alleviation of disorders or symptoms related to a mitochondrial disorder in question.

### REFERENCES

Ahola S, Auranen M, Isohanni P. Niemisalo S, Urho N, Buzkova J, Velagapudi V, Lundborn N, Hakkarainen A, Muurinen T et al (2016) Modified atkins diet induces subacute selective ragged-red-fiber lysis in mitochondrial myopathy patients. EMBO Mol Med 8: 1234-47.
Askanas V, Engel W K, Nogalska A (2015) Sporadic inclusion-body myositis: a degenerative muscle disease associated with aging, impaired muscle protein homeostasis and abnormal mitophagy. Biochim Biophys Acta—Mol Basis Dis 1852: 633-43.
Bender A, Krishnan K J, Morris C M, Taylor G A, Reeve A K, Perry R H, Jaros E, Hersheson J S, Betts J, Klopstock T, Taylor R W, Turnbull D M. High levels of mitochondrial DNA deletions in substantia nigra neurons in aging and Parkinson disease. Nat Genet. 2006 May; 38(5):515-7.
Brownlee M (2001) Biochemistry and molecular cell biology of diabetic complications. Nature 414: 813-20.
Gorman G S, Chinnery P F, DiMauro S, Hirano M, Koga Y, McFarland R, Suomalainen A, Thorburn D R, Zeviani M, Turnbull D M (2016) Mitochondrial diseases. Nat Rev Dis Prim 2: 16080.
Goto, Yu-ichi, Ikuya Nonaka, and Satoshi Horai. 1990. “A Mutation in the tRNALeu(UUR) Gene Associated with the MELAS Subgroup of Mitochondrial Encephalomyopathies.” Nature 348 (6302): 651-53. doi:10.1038/348651a0
Haas R H, Parikh S, Falk M J, Saneto R P, Wolf N I, Darin N, Wong L J, Cohen B H, Naviaux R K (2008) The in-depth evaluation of suspected mitochondrial disease. Mol Genet Metab 94: 16-37.
Hakonen A H, Goffart S, Marjavaara S, Paetau A, Cooper H, Mattila K, Lampinen M, Sajantila A, Lonnqvist T, Spelbrink J N et al (2008) Infantile-onset spinocerebellar ataxia and mitochondrial recessive ataxia syndrome are associated with neuronal complex i defect and mtdna depletion. Hum Mol Genet 17: 3822-35.
Hakonen, A H, S Heiskanen, V Juvonen, I Lappalainen, P T Luoma, M Rantamaki, G V Goethem, et al. 2005. “Mitochondrial DNA Polymerase W748S Mutation: A Common Cause of Autosomal Recessive Ataxia with Ancient European Origin.” Am J Hum Genet 77 (3): 430-41. doi:10.1086/444548.
Khan, Nahid A., Mari Auranen, Ilse Paetau, Eija Pirinen, Liliya Euro, Saara Forsström, Lotta Pasila, et al. 2014. “Effective Treatment of Mitochondrial Myopathy by Nicotinamide Riboside, a Vitamin B3.” EMBO Molecular Medicine 6 (6): 721-31. doi:10.1002/emmm.201403943.
Khan, Nahid A., Joni Nikkanen, Shuichi Yatsuga, Christopher Jackson, Liya Wang, Swagat Pradhan, Riikka Kivelä, Alberto Pessia, Vidya Velagapudi, and Anu Suomalainen. 2017. “mTORC1 Regulates Mitochondrial Integrated Stress Response and Mitochondrial Myopathy Progression.” Cell Metabolism 26 (2): 419-428.e5. doi:10.1016/j.cmet.2017.07.007.
Kley R A, Tarnopolsky M A, Vorgerd M (2013) Creatine for treating muscle disorders. Cochrane Database Syst Rev June 5: CD004760.
Koenig M K, Emrick L, Karaa A, Korson M, Scaglia F, Parikh S, Goldstein A (2016) Recommendations for the management of strokelike episodes in patients with mitochondrial encephalomyopathy, lactic acidosis, and strokelike episodes. JAMA Neurol 73: 591-94.
Koga Y, Akita Y, Nishioka J, Yatsuga S, Povalko N, Tanabe Y, Fujimoto S, Matsuishi T (2005) L-arginine improves the symptoms of strokelike episodes in melas. Neurology 64: 710-12.
Kolho, K.-L., Pessia, A., Jaakkola, T., De Vos, W. M., & Velagapudi, V. (2017). Faecal and Serum Metabolomics in Paediatric Inflammatory Bowel Disease. Journal of Crohn's and Colitis, 321-334.
Kraytsberg Y, Kudryavtseva E, McKee A C, Geula C, Kowall N W, Khrapko K. Mitochondrial DNA deletions are abundant and cause functional impairment in aged human substantia nigra neurons. Nat Genet. 2006 May; 38(5):518-20.
Lehtonen, Jenni M., Saara Forsström, Emanuela Bottani, Carlo Viscomi, Olivier R. Baris, Helena Isoniemi, Krister Höckerstedt, et al. 2016. “FGF21 Is a Biomarker for Mitochondrial Translation and mtDNA Maintenance Disorders.” Neurology 87 (22): 2290-99. doi:10.1212/WNL.0000000000003374.
Lindgren U, Lindberg C, Oldfors A (2017) P.189—incidence and prevalence of inclusion body myositis in western sweden. Neuromuscul Disord 27: S153.
Luoma, P, A Melberg, J O Rinne, J A Kaukonen, N N Nupponen, R M Chalmers, A Oldfors, et al. 2004. “Parkinsonism, Premature Menopause, and Mitochondrial DNA Polymerase Gamma Mutations: Clinical and Molecular Genetic Study.” Lancet 364 (9437): 875-82. doi:10.1016/S0140-6736(04)16983-3.
Morava E, van den Heuvel L, Hol F, de Vries M C, Hogeveen M, Rodenburg R J, Smeitink J A (2006) Mitochondrial disease criteria: diagnostic applications in children. Neurology 67: 1823-26.
Mudd S H, Wagner C, Luka Z, Stabler S P, Allen R H, Schroer R, Wood T, Wang J, Wong L J (2012) Two patients with hepatic mtdna depletion syndromes and marked elevations of s-adenosylmethionine and methionine. Mol Genet Metab 105: 228-36.
Nandania J, Peddinti G, Pessia A, Kokkonen M, Velagapudi V (2018) Validation and automation of a high-throughput multitargeted method for semiquantification of endogenous metabolites from different biological matrices using tandem mass spectrometry. Metabolites 8: 352468.
Nikali, Kaisu, Anu Suomalainen, Juha Saharinen, Mikko Kuokkanen, Johannes N. Spelbrink, Tuula Lonnqvist, and Leena Peltonen. 2005. “Infantile Onset Spinocerebellar Ataxia Is Caused by Recessive Mutations in Mitochondrial Proteins Twinkle and Twinky.” Human Molecular Genetics 14 (20): 2981-90. doi:10.1093/hmg/ddi328.
Nikkanen, J., S. Forsstrom, L. Euro, I. Paetau, R. A. Kohnz, L. Wang, D. Chilov, et al. 2016. “Mitochondrial DNA Replication Defects Disturb Cellular dNTP Pools and Remodel One-Carbon Metabolism.” Cell Metabolism 23 (4). doi:10.1016/j.cmet.2016.01.019.
Noguera A, Fortuny C, Munoz-Almagro C, Sanchez E, Vilaseca M A, Artuch R, Pou J, Jimenez R (2004) Hyperlactatemia in human immunodeficiency virus-uninfected infants who are exposed to antiretrovirals. Pediatrics 114: e598-603.
Oldfors A, Moslemi A R, Fyhr I M, Holme E, Larsson N G, Lindberg C (1995) Mitochondrial dna deletions in muscle fibers in inclusion body myositis. J Neuropathol Exp Neurol 54: 581-87.
Spelbrink, J N, F Y Li, V Tiranti, K Nikali, Q P Yuan, M Tariq, S Wanrooij, et al. 2001. “Human Mitochondrial DNA Deletions Associated with Mutations in the Gene Encoding Twinkle, a Phage T7 Gene 4-like Protein Localized in Mitochondria.” Nat Genet 28 (3): 223-31. doi:10.1038/90058.
Suomalainen, A., J. M. Elo, K. H. Pietilainen, A. H. Hakonen, K. Sevastianova, M. Korpela, P. Isohanni, et al. 2011. “FGF-21 as a Biomarker for Muscle-Manifesting Mitochondrial Respiratory Chain Deficiencies: A Diagnostic Study.” The Lancet Neurology 10 (9). doi:10.1016/51474-4422(11)70155-7.
Suomalainen, A, A Majander, H Pihko, L Peltonen, and A C Syvanen. 1993. “Quantification of tRNA3243(Leu) Point Mutation of Mitochondrial DNA in MELAS Patients and Its Effects on Mitochondrial Transcription.” Hum Mol Genet 2 (5): 525-34.
Tadiboyina V T, Rupar A, Atkison P, Feigenbaum A, Kronick J, Wang J, Hegele R A (2005) Novel mutation in dguok in hepatocerebral mitochondrial dna depletion syndrome associated with cystathioninuria. Am J Med Genet A 135: 289-91.
Thabet H, Brahmi N, Amamou M, Ben Salah N, Hedhili A (2000) Hyperlactatemia and hyperammonemia as secondary effects of valproic acid poisoning. Am J Emerg Med 18: 508.
Thauvin-Robinet C, Faivre L, Barbier M L, Chevret L, Bourgeois J, Netter J C, Grimaldi M, Genevieve D, Ogier de Baulny H, Huet F et al (2004) Severe lactic acidosis and acute thiamin deficiency: a report of 11 neonates with unsupplemented total parenteral nutrition. J Inherit Metab Dis 27: 700-704.
Thompson Legault J, Strittmatter L, Tardif J, Sharma R, Tremblay-Vaillancourt V, Aubut C, Boucher G, Clish C B, Cyr D, Daneault C et al (2015) A metabolic signature of mitochondrial dysfunction revealed through a monogenic form of leigh syndrome. Cell Rep 13: 981-89.
Thorburn D R (2004) Mitochondrial disorders: prevalence, myths and advances. J Inherit Metab Dis 27: 349-62.
Winterthun S, Ferrari G, He L, Taylor R W, Zeviani M, Turnbull D M, Engelsen B A, Moen G, Bindoff L A (2005) Autosomal recessive mitochondrial ataxic syndrome due to mitochondrial polymerase mutations. Neurology 64: 1204-8.
Xia, J, N Psychogios, N Young, and D S Wishart. 2009. “MetaboAnalyst: A Web Server for Metabolomic Data Analysis and Interpretation.” Nucleic Acids Res 37 (Web Server issue): W652-60. doi:10.1093/nar/gkp356.
Xia, J, I V Sinelnikov, B Han, and D S Wishart. 2015. “MetaboAnalyst 3.0—Making Metabolomics More Meaningful.” Nucleic Acids Res 43 (W1): W251-7. doi:10.1093/nar/gkv380.

1. A method for determining a mitochondrial disorder of a subject or predicting a prognosis of a subject having a mitochondrial disorder, wherein the method comprises determining at least four biomarkers sorbitol, alanine, myoinositol and cystathionine from a sample of a subject.

2. A method of selecting a treatment for a subject having a mitochondrial disorder or following up a treatment of a subject having a mitochondrial disorder, wherein the method comprises determining at least four biomarkers sorbitol, alanine, myoinositol and cystathionine from a sample of a subject.

3. The method of claim 1, wherein an elevated or increased level of at least one, two, three or four of the biomarkers selected from the group consisting of sorbitol, alanine, myoinositol and cystathionine in the sample of the subject indicates the mitochondrial disorder and/or prognosis of said subject.

4. The method of claim 1 wherein in the following up the treatment said treatment has positive effects if a level of at least one, two, three or four of the biomarkers sorbitol, alanine, myoinositol and cystathionine decreases after or during said treatment; and/or wherein elevation or increase of one, two or three of the biomarkers sorbitol, alanine, myoinositol and cystathionine indicates a better prognosis compared to a situation wherein at least all four of said biomarkers are elevated or increased in a sample of a subject.

5. The method of claim 1, wherein levels of four biomarkers sorbitol, alanine, myoinositol and cystathionine in the sample of the subject are compared to the levels of said four biomarkers in a control sample or the levels of said four biomarkers in the sample of the subject are compared to the normal levels of said four biomarkers determined from a set of controls.

6. The method of claim 1, wherein the method further comprises determining one or more biomarkers selected from the group consisting of FGF21, GDF15, lactate and pyruvate and any combination thereof, such as FGF21 and GDF15.

7. The method of claim 1, wherein said mitochondrial disorder is a primary or secondary mitochondrial disorder.

8. The method of claim 1, wherein the secondary mitochondrial disorder is an inclusion body myositis (IBM) or Parkinson's disease.

9. The method of claim 1, wherein the primary mitochondrial disorder is a dysfunction affecting the skeletal muscle, heart, central and peripheral nervous system, liver, kidney, and/or the sensory organ systems.

10. The method of claim 1, wherein the primary mitochondrial disorder is selected from the group consisting of mtDNA expression disorders: mitochondrial myopathy, mitochondrial cardiomyopathy, mitochondrial encephalopathy, mitochondrial hepatopathy, mitochondrial renal disease, mitochondrial intestinal disease, mitochondrial blood disease, mitochondrial DNA translation disease, mitochondrial DNA deletion disease, mitochondrial DNA depletion syndrome, infantile-onset spinocerebellar ataxia (IOSCA), mitochondrial recessive ataxia syndrome (MIRAS), progressive external ophthalmoplegia (PEO), chronic progressive external ophthalmoplegia (CPEO), myoclonic epilepsy and ragged-red fibers (MERRF), Kearns-Sayre syndrome (KSS), and a defect of mitochondrial translation such as mitochondrial encephalomyopathy, lactic acidosis and stroke-like episodes (MELAS) or maternally inherited diabetes and deafness (MIDD), including non-symptomatic carriers of disease alleles.

11. The method of claim 1, wherein the sample is selected from the group consisting of a blood sample, plasma sample, serum sample, cheek tissue sample, urine sample, faeces sample, sputum sample, saliva sample, skin sample, muscle sample, cerebrospinal fluid, bone marrow, exhaled air sample, and any tissue or organ biopsy; most specifically the sample is a blood sample.

12. The method of claim 1, wherein the sensitivity of the method to find mitochondrial diseases is more than 60%, 65%, 70%, 75% or 80%, and/or the specificity is more than 70%, 75%, 80%, 85% or 90%.

13. A kit for determining a mitochondrial disorder, predicting a prognosis of a subject having a mitochondrial disorder, selecting a treatment for a subject having a mitochondrial disorder or following up a treatment of a subject having a mitochondrial disorder, wherein said kit comprises tools for determining four biomarkers sorbitol, alanine, myoinositol and cystathionine from a sample of a subject, and optionally reagents for performing a test.

14. The kit of claim 13, wherein the kit further comprises tools for determining one or more biomarkers selected from the group consisting of FGF21, GDF15, lactate and pyruvate and any combination thereof, such as FGF21 and GDF15.

15. The kit of claim 13, wherein the kit comprises tools for an enzymatic assay and/or immunoassay, such as an ELISA assay.

16. The kit of claim 13, wherein the sample is selected from the group consisting of a blood sample, plasma sample, serum sample, cheek tissue sample, urine sample, faeces sample, sputum sample, saliva sample, skin sample, muscle sample, cerebrospinal fluid, bone marrow, exhaled air sample, and any tissue or organ biopsy.

17. The kit of claim 13, wherein said kit is for the method of claim 1.

18. The use of the kit of claim 13 for determining a mitochondrial disorder of a subject, predicting a prognosis of a subject having a mitochondrial disorder, selecting a treatment for a subject having a mitochondrial disorder or following up a treatment of a subject having a mitochondrial disorder.

19. The use of at least four biomarkers sorbitol, alanine, myoinositol and cystathionine for determining a mitochondrial disorder of a subject, predicting a prognosis of a subject having a mitochondrial disorder, selecting a treatment for a subject having a mitochondrial disorder or following up a treatment of a subject having a mitochondrial disorder.
